(19) United States
(12) Patent Application Publication
(10) Pub. No.: US 2023/0263119 A1
(43) Pub. Date: Aug. 24, 2023

(54) RESISTANCE TO FUNGI

(71) Applicant: ORIGENE SEEDS LTD, Rehovot (IL)

(72) Inventor: Eyal A. VARDI, Givat Brenner (IL)

(21) Appl. No.: 18/024,059

(22) PCT Filed: Aug. 22, 2021
(86) PCT No.: PCT/IL2021/051027

§ 371 Date: Mar. 1, 2023

Related U.S. Application Data
(60) Provisional application No. 63/074,437, filed on Sep. 3, 2020.

(51) Int. Cl. A01H 1/00; A01H 1/02; A01H 1/04; A01H 6/34

(52) U.S. Cl. CPC A01H 1/1255; A01H 1/021; A01H 1/045; A01H 6/342

(57) ABSTRACT

A watermelon plant and seed, namely of Citrullus lanatus, which are resistant to fungi, including Watermelon Powdery Mildew (PM), comprising in their genome introgressed sequences from Citrullus lanatus var. Citroides conferring resistance to the fungi. More specifically, the invention also discloses molecular genetic markers, linked to the dominant genetic locus conferring resistance to fungi characterized by a resistance to powdery mildew (PM) race 1 and race 2 as quantified by a tolerance of less than about 3.5 on the Tetteh scale, and having properties such that seedless fruits are produced with a total soluble solid (TSS) of at least 10%, flesh firmness of between 60 and 80 on a scale of 10 to 90, where 10 indicates very soft flesh and 90 indicates a very firm flesh, and a flesh color selected from the group consisting of yellow, orange or red fruit flesh color, preferably dark red.

### FIELD OF THE INVENTION

The invention concerns watermelon plants with resistance to fungal diseases and to methods for their obtaining. More specifically, the present invention is directed to watermelon plant and seed, namely of Citrullus lanatus, which are resistant to fungi, including Watermelon Powdery Mildew (PM), comprising in their genome introgressed sequences from Citrullus lanatus var. Citroides conferring resistance to said fungi. More specifically, the invention also discloses molecular genetic markers, linked to the dominant genetic locus conferring resistance to fungi.

### PRIOR ART

References considered to be relevant as background to the presently disclosed subject matter are listed below:
Davis A R, Tetth A, Whner T, Levi A, Pitrat M, 2006. Watermelon resistance to powdery mildew race 1 and race 2. In G. J. Holmes (ed.) Proc. Cucurbitaceae 2006. Universal Press, Raleigh, N.C.; 412-420.
Levi A, Thomas C E, Trebish T, Salman A, King J, Karalius J, Newman M, Reddy O U K, Xu Y, Zhang X, 2006. An extended linkage map for watermelon based on SRAP, AFLP, SSR, ISSR and RAPD markers. J Amer. Soc. Hort. Sci. 131(3): 393-402
Levi A, Claude E T, 2007. DNA markers from different linkage regions of watermelon genome useful in differentiating among closely related watermelon genotypes. HortScience 42(2): 210-214.
Pitrat M, Besombes D, 2008. Inheritance of Podosphaera xanthii resistance in melon line ‘90625’. Cucurbitacaea 2008, Proceeding of the IXth EUCAPRIA meeting on genetics and breeding of Cucurbitaceae, 135-142.
Robinson R W. Provvidenti R, Shail W, 1975. Inheritance of susceptibility to powdery mildew in the watermelon. The J of Heredity 66: 310-311.
Tetteh A Y, Whener T C, Davis A R, 2010. Identifying resistance to powdery mildew race 2W in the USDA-ARS watermelon germplasm collection. Crop Sci. 50: 933-393.
Zhang H, Gou S, Gong G, Ren Y, 2011. Sources of resistances to race 2WF powdery mildew in US watermelon plant introductions. HortScience, 46(10): 1349-1352.

### BACKGROUND OF THE INVENTION

Watermelon (Citrullus lanatus), family Cucurbitaceae, is a major cucurbit crop and includes both seeded and seedless fruits.

During the last 50 years, the world production area of watermelon has increased with 62%, from 1.96 million ha in 1961 to 3.16 million ha in 2012. The yield tripled during the same period, resulting in a fivefold total production increase (from 17.8 to 89.0 million tons). The top 10 world watermelon producers include China, accounting for 63% of the production in 2010, followed by Turkey, Iran, Brazil, USA, Egypt, Uzbekistan, Russia, Mexico and Algeria (FAOSTAT 2012 in Jensen, 2012, Cucurbitaceae 2012 p. 264-273). In the United States, watermelon is major vegetable crop. Farmers in 44 States grow more than $500 million worth of watermelon commercially every year (USDA, 2017). Major production states are Florida, California, Arizona, Texas and Georgia. In 2005, the total production of watermelon in the United States was 1.7 million Kg, with a farm value of 410 million US$ (USDA, 2006 IN Tetteh et al., 2010, Crop Sci. 50:933-939).

In Turkey, watermen production in 2012 was 4 million tons (165,000 hectare).
In Iran, watermen production in 2012 was 3.8 million tons (145,000 hectare).
In Brazil watermen production in 2012 was 2 million tons (95,000 hectare).

The watermelon fruits have varying size ranging from very big watermelon, at times >7 Kg to medium, 4-7 kg and small watermelons (<4 kg, known as the mini or personal size). The red pigment in red—fleshed watermelon is from the carotenoid lycopene and comprises 70-90% of the total carotenoids in watermelon.

In recent years watermelon has been characterized also at the DNA level and DNA markers from different linkage regions of the watermelon genome have been identified using various PCR reaction techniques [Levi A et al., 2006, Levi A et al., 2007].

Powdery mildew (PM) is one of the most common diseases of watermelons. In the last years, the PM disease spread worldwide and become a significant threat [Zhang et al., 2011]. Two species Podosphaera xanthii (formerly Sphaerotheca fulginea) and Golovinomyces cichoracearum (formerly Erysiphe cichoraearum), can induce worldwide typical and identical symptoms [Pitrat & Besombes, 2008].

The PM disease is spread both in open fields and in greenhouses. In open fields the disease is usually limited to dry areas or to dry seasons.

The first appearance of the disease is being recognized as white colony, 2-3 mm diameter, appearing on the cotyledons or on the leaves. With time, the colonies grow, unite and create continues cover of white mycelium and white spores on both sides of the leaves, which looks like powder. Spores number can reach up to 50-100,000 per 1 cm².

Later colonies may appear on the stems and even on the fruits. Necrosis starts a week after the infection, depends on environment conditions. Under optimal growth conditions of the pathogen and without a suitable treatment, the fungus can cause death of the whole plant in a couple of weeks. The spores' germination process occurs in drought conditions. Presence of water on the tissue prevents the germination. The optimal temperature for germination is 20° C.-23° C. (minimum 5° C., maximum 35° C.). Intensive photosynthesis improves the germination.

Watermelons were resistant to older races of Sphaerotheca fuliginea present in the U.S. in the 1970s. However, in recent years, a single recessive gene pm having high susceptibility to PM was found in the Plant Introduction, PI 269677 [Robinson et al., 1975].

Currently, two races of PM are found in the U.S. named race one (PM1W) and race two (PM2W), and induce a susceptible reaction in most cultivars. In the past, watermelon was considered to be free of powdery mildew. In recent years, powdery mildew outbreaks have been reported (also) in the United States. The disease has been confirmed in South Carolina, Georgia, Florida, Maryland, Texas, Oklahoma, Arizona, New York and Carolina. 1654 Citrullus cultigens from the U.S. plant introduction collection were evaluated for resistance to PMW2. 93% of the cultigens had total plant disease severity rating of >4.0 (sensitive), which means at least 20% mycelium coverage on leaves and stems. 7% of the cultigens had high resistance (<3.0) or intermediate resistance (3.1-4) [Tetteh et al., 2010, Davis et al., 2006].

PM1W usually appears at the beginning of a season where the plant is still considered a young plant, while PM2W usually appears late in the season, after fruit setting.

The major damages of PM on watermelon are decrease in the yield quality, namely decreased fruit size and number of fruits per plant. The fruits which remain have poor fruit quality and poor flavor and experience short storage life.

It has been previously reported that PM can be controlled with fungicides. However, resistance to the recommended fungicides, especially the strobilurins and myclobutanil was reported. Also the disease control often requires the use of systemic fungicide because spray application to the underside of leaves is difficult. Currently, effective control of powdery mildew is achieved with alternating preventative applications of mancozeb with azoxystrobin [Tetteh et al., 2010].

Therefore, there is a need for an effective and safe method to control PM while minimizing the use of chemicals.

### SUMMARY OF DISCLOSURE

The present disclosure provides, in accordance with a first of its aspects, a triploid hybrid watermelon (Citrullus lanatus) plant characterized by resistance to powdery mildew (PM) and capable of producing seedless fruits with a total soluble solid in the range of 10% to 13%, and a red fruit flesh color.

In accordance with a second of its aspects, the present disclosure provides seeds of a triploid watermelon plant characterized by resistance to powdery mildew (PM) and capable of producing seedless fruits with a total soluble solid in the range of 10 to 13%, and a red fruit flesh color.

Further, there is provided by a third aspect of the present disclosure a diploid watermelon line having at least tolerance, at time at least intermediate resistance to PM and being capable of pollinating a tetraploid watermelon to produce a triploid hybrid watermelon with resistance to powdery mildew (PM) and capable of producing seedless fruits with a total soluble solid in the range of 10% to 13%, and a red fruit flesh color, as disclosed herein.

In accordance with further aspects of the present disclosure there are provided pollen and seeds of the diploid watermelon line disclosed herein.

In accordance with another aspect of the present disclosure there is provided a method of producing a triploid hybrid watermelon plant characterized by resistance to powdery mildew (PM) and capable of producing seedless fruits with a total soluble solid in the range of 10% to 14%, at times in the range of 11% to 13.5%, and a red fruit flesh color, the method comprising:
a. planting a PM sensitive female parent tetraploid watermelon line having a total soluble solids of at least 10%, essentially red fruit flesh color, flesh firmness of between 60 to 80 and thousand seeds weight (TSW) of between 25 to 90 g;
b. pollinating said tetraploid watermelon line with pollen from a male parent diploid watermelon line having at least tolerance to PM;
c. planting said triploid watermelon seeds to produce said triploid watermelon plants.

Further provided herein is a method of producing a triploid hybrid watermelon fruit harvested from a plant with resistance to powdery mildew (PM) and the fruit being seedless, with a total soluble solid in the range of 10% to 14%, at times within the range of 11% to 13.5% and at times about 13.5%, and a red fruit flesh color and, the method comprising:
a. planting a PM sensitive female parent tetraploid watermelon line having a total soluble solid of at least 10%, red fruit flesh color, flesh firmness of between 60 to 80 and TSW of between 25 to 90 g;
b. pollinating said female tetraploid watermelon line with pollen from a male parent diploid watermelon line having at least tolerance to PM;
c. planting said triploid watermelon seeds to produce said triploid watermelon hybrid plants and exposing said triploid hybrid plant to a pollinizer to produce said triploid seedless watermelon fruit;
d. harvesting triploid seedless watermelon fruits from said triploid hybrid watermelon plants.

In some embodiments, the diploid watermelon male parent is one identifiable by a marker locus which co-segregates with PM resistance trait, the marker locus being identified by a PCR reaction comprising amplification of a DNA fragment with a pair of PCR oligonucleotide primers represented by a forward primer 5′ to 3′ of SEQ ID NO:1 (TGAGTCCAAACCGGATA, also known as me1) and a reverse primer 5′ to 3′ of SEQ ID NO:2 (GACTGCGTACGAATTAAT also known as em1) or any other marker located on same chromosome that is statistically correlated and genetically linked to the PM resistance trait.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1A-1D are images of PM sensitive watermelon plant infected with white mycelium spores appearing on the leaves (FIG. 1A); on the stems (FIG. 1B) and even on the fruits (FIG. 1C); which may result in death of the whole plant in a couple of weeks (FIG. 1D).

FIG. 1E is an image of PM tolerant diploid male plants (left) and PM sensitive diploids male plants (right), growing in a greenhouse in southern of Israel. The plants were grown in greenhouse and were inoculated by spreading uniformly the fungus by hand in the greenhouse. Also, the plants in the greenhouse were not treated with fungicides.

FIGS. 2A-2D are images of the wild type PM resistant watermelon PI 482312 showing the fruits striped rind (FIG. 2A), yellow flesh (FIG. 2B), green seeds (FIG. 2C) and PM tolerant leaves (FIG. 2D).

FIG. 3 is an image of a triploid watermelon hybrid according to the present disclosure after exposure to PMW1 and PMW2 (area marked “I” in the field) as compared to a reference seedless watermelon (area marked “II” in the field).

FIG. 4 is an image of leaves of a PM tolerant seedless watermelon according to the present disclosure after exposure to PMW1 and PMW2 (marked by a full arrow) as compared to an infected PM sensitive reference seedless watermelon (marked by a dashed arrow).

FIGS. 5A and 5B are images of a watermelon fruit of a diploid male parent during selection stages, FIG. 5A showing a fruit of a diploid male parent at intermediate flesh and rind uniformity stage and FIG. 5B at a more progressed flesh and rind uniformity and quality stage.

### DETAILED DESCRIPTION OF EMBODIMENTS

Generally, the present disclosure is based on the unique development of a triploid hybrid watermelon plant lines with agriculturally acceptable resistance to powdery mildew and capable of producing edible seedless fruits. These triploid hybrid watermelon plant lines are the subject of the first aspect of the present disclosure.

By a second aspect, there are provided diploid male inbred watermelon lines with at least tolerance, at times at least intermediate resistance to PM and tetraploid female inbred watermelon lines having red flesh color and total soluble solids of at least 10%, which when crossed, provide the triploid watermelon lines capable of producing seedless fruits with a TSS in the range of 10% to 14%, at times in the range of 11% to 13.5%.

Also provided herein are methods of use of the watermelon lines, as further discussed below.

In the present the following terms are to be understood as having the following meaning:

Powdery mildew (PM)—In the context of the present disclosure when referring to Powdery mildew (PM) it is to be understood as having the meaning known in the art, i.e. a fungal disease characterized by spots or patches of white to grayish, talcum-powder-like growth. As appreciated, different types of PM are differentiated using various melon differentials. When referring to watermelon, PM refers to sensitivity of a watermelon plant to at least one of PM race 1 and race 2 (hereinafter “PMW1” and “PMW2”, respectively).

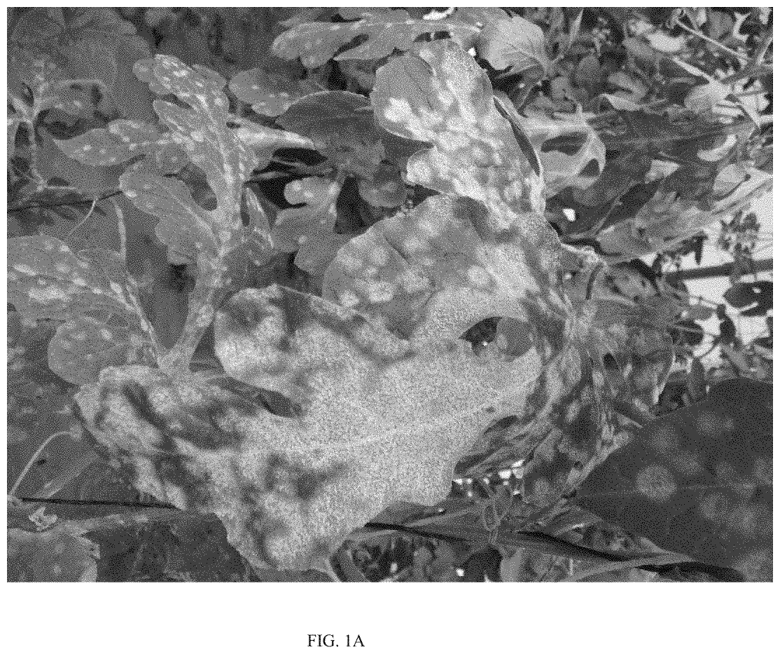

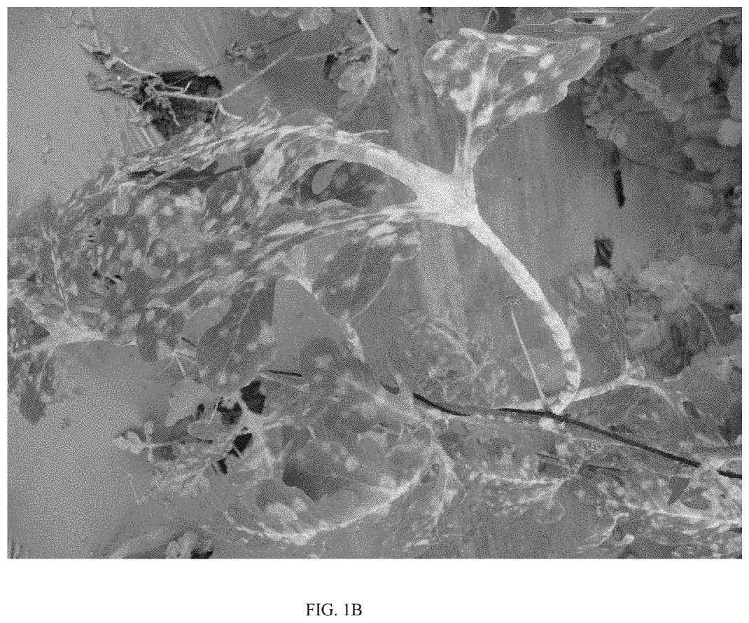

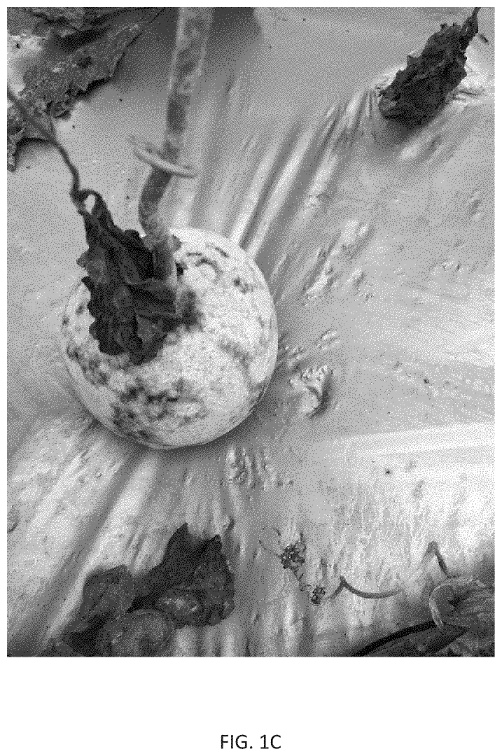

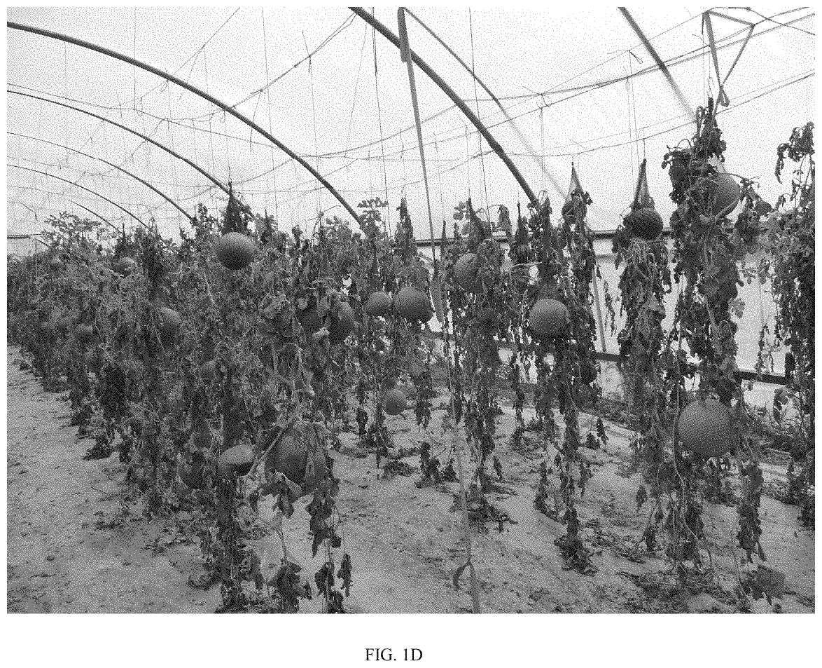

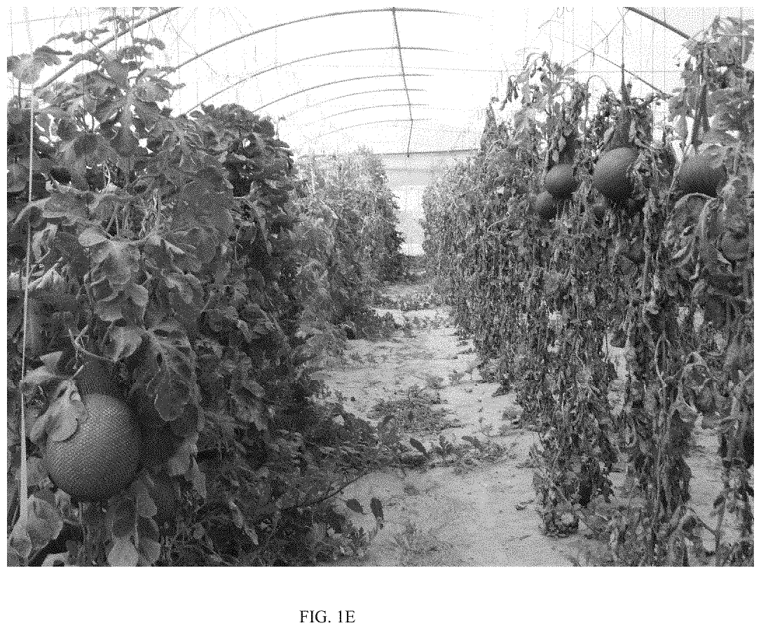

The sensitivity of a plant is determined according to the size of mycelium and time of its appearance on the plant during the cultivation period. Typically, the first appearance of the disease is recognized as white colony, 2-3 mm diameter, appearing on the cotyledons or on the leaves. With time, the colonies grow, unite and create continues cover of white mycelium and white spores on both sides of the leaves, which looks like powder. FIG. 1A shows leaves of a PM sensitive watermelon infected with white mycelium spores. Spores number can reach up to 50-100,000 per 1 cm². Later colonies may appear on the stems (FIG. 1B) and even on the fruits (FIG. 1C). Necrosis starts a week after the infection, depending on environment conditions. Under optimal growth conditions of the pathogen and without a suitable treatment, the fungus can cause death of the whole plant in a couple of weeks (FIG. 1D). The spores germination process occurs in drought conditions. Presence of water on the tissue prevents the germination. The optimal temperature for germination is 20-23° C. (minimum 5° C., maximum 35° C.). Intensive photosynthesis improves the germination.

Tolerance or resistance—tolerance (or lack of sensitivity) is ranked between 1 to 9, with 1 defines a high degree of sensitivity (no tolerance) that even results in necrosis, and 9 defining high tolerance or even up to resistance. Thus, it is be understood that a sensitive plant has low tolerance to PM and lack of sensitivity is indicative of tolerance or resistance to PM. Accordingly, a plant ranked between 5-6 is regarded as tolerant, a plant ranked 6-8 is regarded high tolerance or intermediate resistance, and a plant ranked 8-9 is regarded as resistant. In some embodiments, when referring to tolerance, intermediate resistance or resistance, it is to be understood as referring to a plant comprising a resistance locus being linked to a genetic background or genetic determinant obtainable from the genome of a wild type watermelon plant being agriculturally recognized as having resistance to PM (scale of resistance above 7, and preferably above 8), such as the PM watermelon plant designated according to the plant index PI 482312 [Tetteh et al. 2010]. A comparison between PM sensitive line (ranked as 2) and PM tolerant line (ranked as 8) can be seen in FIG. 1E (on the left is the PM tolerant and on the right is the sensitive line).

Seedless—In the context of the present disclosure it is to be understood that a seedless plant is defined by lack of mature seeds in its fruit and thus lacking the capacity to propagate via seeds. It is to be understood that a seedless fruit may include seed coats and with respect to the present invention a seedless fruit is one comprising between 0-200 seed coats.

Fruit shape—Generally, a watermelon fruit may have a flat, round, oval and elongated shape. The shape may be defined by the ratio between the major and minor axes of the fruit, such that an oval shape is defined by a ratio between the major axis 1.2-1.4 to the minor axis 1 as a ratio between 1.2:1 to 1.4:1 while in an elongated fruit the ratio is define between 1.5:1 and 1.8:1. Equally the shape may be scaled between 10 to 90, with 10 representing a flat fruit, 50 round fruit, 60-70 represents an oval fruit and 75-90 represents an elongated fruit. This scale is calculated based on the fruit's measurements ratio (length/width)×50 (a round fruit).

Fruit regularity—may be ranked between 10 to 90, where 10 indicates an irregular shape and 90 indicates that the fruit is regular in shape.

The term “Fruit shape” as used herein shall include but will not be limited to a flat, round, oval and elongated shape. The shape may be defined by the ratio between the major and minor axes of the fruit, such that an oval shape is defined by a ratio between the major axis 1.2-1.4 to the minor axis 1 as a ratio between 1.2:1 to 1.4:1 while in an elongated fruit the ratio is define between 1.5:1 and 1.8:1. Equally, the shape may be scaled between 10 to 90, with 10 representing a flat fruit, 50 round fruit, 60-70 represents an oval fruit and 75-90 represents an elongated fruit. This scale is calculated based on the fruit's measurements ratio (length/width)×50 (a round fruit). Thus, in some embodiments, the fruit shape of the watermelon fruits disclosed herein is between 50 to 70, e.g. ranging from round fruit to oval fruit.

The term “Small fruit size” as used herein shall include but will not be limited to a fruit are typically weighting less than 4.0 kg, at times, between 1.5 and 4.0 kg and at times, between 2 Kg-4 Kg, and are recognized as the mini or personal sized watermelons.

The term “Midi fruit size” as used herein shall include but will not be limited to a fruit weighing 4 Kg-7 Kg.

The term “Large fruit size” as used herein shall include but will not be limited to a fruit weight and at least 7 Kg.

Vigor—watermelon plants may be characterized by their overall condition or their “vigor”, determined based on plant size and leaf blade size.

The term “Vigor” as used herein shall include but will not be limited to a health condition, a hardiness indication, a measure of the increase in plant growth or foliage volume through time after planting, a measurement indicator including biomass production, a number of new shoots produced, a number of reproductive shoots produced, a plant height, a plant volume, a plant height, a plant basal diameter, a number of leaves, a number of stems, a number of leaf whorls, a diameter of rosette, and a volume of plant (height×cover). Although vigor indicators are also strongly influenced by annual weather patterns, they may be appropriate for some monitoring questions. As nondestructive measures of vigor, they are appropriate for use during the plant growth cycle. Most are easy to measure, with little observer bias.

Vigor characteristics of watermelon plants may be defined by their overall condition or their “vigor” or “strength” as commonly determined in botany (e.g. a measure of an increase in plant growth or foliage volume through time after planting), the vigor being scaled between 10 to 90 on a vigor scale; 10 defining a weak vigor/weak plant and score of “90” on the vigor scale, defining a very strong vigor. Vigor is determined based on plant size and leaves blade size. In some embodiments, vigor of the tolerant plants is between 50-90 on the vigor scale, while vigor of sensitive plant decrease to 10-45 on the vigor scale.

Flowering period—flowering period, also understood as earliness of a plant defined by the time from planting to the first female flower and time from flowering to fruit maturation. In accordance with the present disclosure, earliness, i.e. the female flowering time is scaled between 10 to 90; 10 defining a very early female flowering time and 90 defining a very late female flowering time. In the summer, time to early (10) female flowering is defined between 20-25 days after planting and late (90) is defined 40-45 days after planting.

Fruit setting—the occurrence of fruits setting in a plant, and in the context of the present disclosure, the setting is scaled between 10 to 90; 10 reflecting very few up to 0.5 fruit per plant and 90 reflecting a large fruits number per plant (above 4).

Flower sex—flower may be monoecious (M), namely, a watermelon plant species in which male and female organs are found on the same plant but in different flowers, hermaphrodite (H), namely, a plant where the male and female organs are present on the same flower; and andromonoecious (AM), namely, a plant that has both hermaphrodite and male flowers.

Rind color—the color of the rind of the fruit may be from smooth gray, to striped fruit like brake green stripe (citron type), thin stripes (Jubilee/Tiger type), dark medium stripes (crimson type) and dark wide stripes on light green background (All sweet type) to smooth light or dark green rind.

Number of fruit per plant—the number of fruit per plant is well appreciated parameters in determining quality of a plant.

Striping—watermelons may be distinguished by the appearance of stripes on the outer surface of the rind. The stripes may cover small portions of the fruit's rind, medium coverage and also may cover the majority of the fruit's rind. The stripes may be very thin, medium or very thick. Thus, the overall stripes coverage of the rind, may be defined by percentage, with low coverage being regarded as less or equal to 10% rind cover, medium coverage—50% cover, and high/thick coverage—70-80% rind cover and very high/thick coverage—90% rind cover. The stripes are typically green stripes.

Weight—watermelon fruits are typically characterized by their weight. Small fruits are those typically weighting less than 4.0 kg, at times, between 1.5 and 4.0 kg and at times, between 2-4 Kg, and are recognized as the mini or personal sized watermelons, and large fruits are those typically weighting between at least 4.0 Kg. The group of large fruits may be further divided into two sub groups of sizes 4. Kg-6 Kg (also referred to as midi sized fruits) and at least 7 Kg. Thus, in the context of the present disclosure, the fruits may be mini, having a weight of less than 4.0 kg, between 2.0 and 4.0, midi, having a weight of between 4.0 and 6 kg, and large fruits having a weight larger than 7 kg, such as between 7-14 kg or 7 kg to 10 kg.

The term “Flesh color” of a watermelon flesh color may vary from scarlet red (dark red), coral red (light red), orange, salmon yellow, canary yellow, or white. In the context of the present disclosure, when referring to red it is to be understood to cover also shades or hues of red, including dark red or red like color. The flesh color may be determined using Pantone Color scale and in accordance with some embodiments, the red color is identified by any one of the Pantone color scale including “red” in their name as available on line at www.pantone.com (2012). In some embodiments, the flesh color is dark red.

Flesh firmness—flesh firmness defines the force necessary to break the flesh tissue at ripening. The term “Flesh firmness” as used herein shall include but will not be limited to a force necessary to break the flesh tissue at ripening. Flesh firmness of the watermelon plants disclosed herein was measured by PENETROMETER FRUIT PRESSURE TESTER mod. FT 011 (0-11 lbs.), IRC using an 11 mm plunger attachment. Flesh firmness measures the pressure necessary to force a plunger of specified size into the pulp of the fruit. Such pressure is measured in either pounds or Kilograms. The scale in accordance with the present disclosure, using an 11 mm plunger attachment, was ranked between 1 to 11 lbs., where 1 lbs. indicates that the flesh is very soft, and 11 lbs. indicates a ultra-firm flesh. 1-2 lbs. indicates soft flesh. 2-3 lbs. indicates regular/standard firm flesh. 3-4 lbs. indicates firm flesh. 5-6 lbs. indicates very firm flesh. 7-11 lbs. indicates ultra-firm flesh. Thus, in some embodiments, the flesh firmness of the drought tolerant watermelon fruit is between about 2.5-3.5.

According to methodology known in the art, the measurement are obtained using specialized tips for each type of fruit being tested—3 mm (berries, grapes and other small fruits), 6 mm (avocados, berries), 8 mm (pears, stone fruit, avocados) and 11 mm (apples). Such pressure is measured in pounds or kilograms. The scale in accordance with the present disclosure was ranked between 10 to 90 (50 being 2 lbs), where 10 indicates that the flesh is very soft (e.g. penetrometer scale below 2), and 90 indicates a very firm flesh (e.g. penetrometer scale above 4). Penetrometer scale between 3-3.5 is considered firm flesh.

Optionally, when using an 8 mm tip, flesh firmness to a weight/pressure was ranked between 3 ounces to 31 ounces, where 3 ounces indicates that the flesh is very soft and 31 ounces indicates a very firm flesh. In some embodiments, the triploid hybrid watermelon disclosed herein, is characterized by fruit flesh firmness to pressure of 20-27 ounces when using an 8 mm tip, at times 24 ounces.

Rind thickness—is usually measured in millimeters from the outer edge of the fruit to the boundary between the white mesocarp and colored endocarp. The thickness may vary from very thick, e.g. >19 mm, medium thickness 10-19 mm, to low thickness <10 mm.

Rind crack—rind crack defines the tendency of the watermelon rind to crack by itself and in accordance with the present disclosure, rind cracks are ranked from 10 to 90, where 10 defines a strong, uneasily cracked rind (no tendency to crack at all) and 90 defines an easily cracked rind (the fruit is cracking by itself in the field).

Taste—while being somewhat a subjective characteristic, a fruit of a watermelon may vary from tasteful, tasteless, bitter etc.

TSS (Total Soluble Solids)—is a measure of the percent soluble solids (TSS) in a sample of a plant juice, used as an index/parameter for sugar quantity in the fruit (sweetness). The TSS was measured by ATAGO refractometer (http://www.atago.net/USA/products_hsr.php). Brix 0.0 to 33.0%, Automatic Temperature Compensation. The refractometer designed to measure the refractive index of the solution. The Brix percentage represents the total concentration of total soluble solids (TSS) in the sample.

The term “TSS” as used herein, shall include but will not be limited to an amount of solids dissolved, an amount of solids dissolved measured according to a brix scale, a percent soluble solids (TSS) in a sample of a plant juice, used as an index/parameter for sugar quantity in the fruit (sweetness). The TSS was measured by ATAGO refractometer (http://www.atago.net/USA/products_hsr.php), Brix 0.0 to 33.0%, Automatic Temperature Compensation, The refractometer designed to measure the refractive index of the solution. The Brix percentage represents the total concentration of total soluble solids (TSS) in the sample. At tines, the TSS correlates with the plant's fruit general taste. Taste, while being somewhat a subjective characteristic, defines whether the fruit is tasteful, tasteless, bitter and the like.

The term “Brix” as used herein shall include but will not be limited to measuring the percentage of Total Soluble Solids (TSS) in a given substance (per 100 grain), a method to identify the naturally ripened (on-vine) from artificially/post-harvest ripened fruits and a sugar content of an aqueous solution (Degrees Brix (symbol ° Bx)) wherein one degree Brix is 1 grain of sucrose in 100 grams of solution and represents the strength of the solution as percentage by mass. If the solution contains dissolved solids other than pure sucrose, then the ° Bx only approximates the dissolved solid content. The ° Bx is traditionally used in the wine, sugar, carbonated bever^{a}ge, fruit juice, maple syrup and honey industries.

Comparable scales for indicating sucrose content are the degree Plato (° P), which is widely used by the brewing industry, and the degree Balling, which is the oldest of the three systems and therefore mostly found in older textbooks, but also still in use in some parts of the world.

By way of an example only, a sucrose solution with an apparent specific gravity (20°/20° C.) of 1.040 would be 999325° Bx or 9.99359 P while the representative sugar body, the International Commission for Uniform Methods of Sugar Analysis (ICUMSA), which favors the use of mass fraction, would report the solution strength as 9.99249% Because the differences between the systems are of little—19—practical significance (the differences are less than the precision of most common instruments) and wide historical use of the Brix unit, modern instruments calculate mass fraction using ICUMSA official forriulas but report the result as ° Bx.

The content (which solids are, dissolved) of solids dissolved, is deterrnined by refractive index by way of using a refractometer and is referred to as the degrees Brix.

Deposit—deposit of representative seeds of the lines referred to by the Applicant's internal reference and having representative seeds deposited on 24 Aug. 2012, at the NCIMB recognized institute for purposes of patent procedure and according to which the following Accession number for the deposition of Citrullus lanatus was provided NCIMB42044, as well as the one deposited in the same NCIMB institute on Nov. 16, 2012 and having the Accession No. NCIMB42083 and the one deposited on Oct. 14, 2013, and having representative seeds deposited under Accession No NCIMB42172, as well as those deposited in the same NCIMB institute on Feb. 16, 2015, referred to by Applicant's internal references 12.702b-W14 and 12.135b-W14 and having, respectively, the Accession Nos. NCIMB42359 NCIMB42360.

Turning now to the present disclosure, there is provided, in accordance with a first aspect, a triploid watermelon hybrid plant characterized by resistance to powdery mildew (PM) and capable of producing seedless fruits with a total soluble solid (TSS) in the range of 10% to 14%, at times 11% to 13.5% and red fruit flesh color.

In accordance with some embodiments, the triploid hybrid watermelon plant has a tolerance rank of at least 6, preferably, at least 7, at times, between 7 to 9, to at least one strain of PM, at times, to at least PMW1 or PMW2 and yet at times, to PMW1 and PMW2. Preferably, a tolerance scale score of 7-9 is achieved as indicative of PMW1 or PMW2 resistance.

When referring to various characteristics of watermelon plants, it is noted that the triploid hybrid watermelon plant disclosed herein may be further characterized by any one or more of the following.

In some embodiments, the triploid hybrid watermelon plant is characterized by vigor of between 50 to 80, at times about 70 (in the scale between 10 to 90).

In some embodiments, the triploid hybrid watermelon plant is characterized by earliness of between 30 to 70, at times between 40 and 60.

In some embodiments, the triploid hybrid watermelon plant is characterized by fruit setting of between 20 to 50.

In some embodiments, the triploid hybrid watermelon plant is characterized by flower sex being monoecious (M), namely, a triploid watermelon plant species in which male and female organs are found on the same plant but in different flowers.

In some embodiments, the triploid hybrid watermelon plant is characterized by rind color of crimson to dark crimson color. In some other embodiments, the plant is characterized by tiger type rind.

In some embodiments, the triploid hybrid watermelon plant is characterized by green stripes on the fruit, and in some embodiments, gray fruit having thick dark green stripes that cover between 60% to 90%, typically about 80% of the fruit.

In some embodiments, the triploid hybrid watermelon plant is characterized by number of fruits per plant in the range of 1 to 4, preferably 2-3.

In some embodiments, the triploid hybrid watermelon plant is characterized by flesh color being scarlet red (dark red) to coral red (light red) according to PANTONE scale color: http://www.pantone.com/pages/pantone/colorfinder.aspx. Dark red (Pantone 18-1664 TCX Fiery Red), light red (Pantone Red 032 C). In some embodiments, the flesh color is dark red. In some other embodiments, the flesh color is light red.

In some embodiments, the triploid hybrid watermelon plant is characterized by flesh firmness between 60 to 80, at times about 70.

In some embodiments, the triploid hybrid watermelon plant is characterized by rind crack of between 10 to 90, typically about 10 to 30, i.e. no or very little (insignificant) tendency to crack.

In some embodiments, the triploid hybrid watermelon plant is characterized by good taste, as determined by a human subject tasting the fruit as being non-bitter.

In some embodiments, the triploid hybrid watermelon plant is characterized by TSS (Brix %) between 10% to 14%, at times between 11% to 13.5% or about 13.5%.

In some embodiments, the triploid hybrid watermelon plant is characterized by resistance to at least PMW1R, at times to at least one of PMW1R and PMW2R.

In some embodiments, the fruit is an essentially round fruit. In some other embodiments, the fruit is oval. The fruit is not regarded as an elongated fruit, i.e. the fruit is scaled between 50 to 70 but not above 70 (which indicative, as described above, of an oval fruit shape).

In some embodiments, the fruit is a mini fruit, having a weight in the range of 2-4.0 kg, preferably between 2.5-3.5 kg or 2.8 kg-3.2 kg and in some other embodiments the fruit is a midi fruit, having a weight between 4 to 6 kg, at times, between 4-5 kg.

In some embodiments the fruit is round and has a weight in the range of 2-4.0 kg, preferably between 2.8-3.2 Kg.

In some embodiments the fruit is oval and has a weight greater than 4.0 kg, at times, between 4 to 6 kg, or even between 6 to 10 kg.

The triploid hybrid watermelon plant disclosed herein carries genetic background from the PM watermelon plant designated according to the plant index PI 482312 [Tetteh et al. 2010] as having tolerance equivalent to tolerance of rank 6 according to the tolerance scale of the present disclosure, or 3.5 according to Tetteh et al., 2010 Comparison between currently used ranking (Numerical Rank/Current) and ranking according to Tetteh et al., 2010 is provided below, with “R” representing Resistant (rank 8-9), “IR” representing Intermediate Resistant or, as used in the present disclosure, Tolerance; and “S” representing susceptibility or sensitivity.

| Numerical | 0 | 1 | 2 | 3 | 4 | 5 | 6 | 7 | 8 | 9 |
|---|---|---|---|---|---|---|---|---|---|---|
| Current | — | S | S | S | S | S | IR | IR | R | R |
| Tetteh et al. 2010 | R | R | R | R* | IR | S | S | S | S | S |

*≤3 represents “R”,
3.1 << 4 represent “IR” (Tetteh et al., 2010)

The winter 2011 offspring (referred to herein as W11-701-1 or PI 482312-PMR) and being originally obtained from self-pollination of this PI from summer 2005.

When referring to “genetic background” in the context of the present invention it is to be understood to refer to a cultivated watermelon plant as disclosed herein (the diploid, tetraploid or the triploid plant) containing a genome comprising at least a qualitative trait locus (QTL) which contributes to PM tolerance.

In some embodiments, the QTL is genetically linked to at least one marker locus that co-segregates with the PM resistance trait and can be identified by a DNA profiling analysis technique, such as, but not limited to, PCR analysis (Polymerase Chain Reaction), SRAP analysis (Sequence-related Amplified Polymorphism) analysis, SSR analysis (Simple Sequence Repeat), AFLP analysis (Amplified Fragment Length Polymorphism) and RAPD analysis (Random Amplified Polymorphic DNA), SNP analysis (Single Nucleotide Polymorphism).

Overall PM—Citrullus lanatus var. Citroides introgression performed is displayed graphically depicting a resistance locus genetically linked to at least one marker locus which co-segregates with PM resistance trait and comprises a marker that has been identified for the resistance to PM fungus is conferred by an introgression of genomic DNA of Citrullus lanatus var. Citroides on the chromosome 2 of Citrullus lanatus. Subsequent to Whole Genome Sequencing (WGS) we performed in 2016 we identified that the resistance to Powdery Mildew fungus was conferred by an introgression of genomic DNA of Citrullus lanatus var. citroides on the 2nd chromosome of Citrullus lanatus. WGS analysis have enabled us to learn that the introgression of Citrullus lanatus var. citroides into the Citrullus lanatus genome is between nucleotides 24,000,000-28,000,000. Surprisingly, farther WGS enabled us identified that the resistance to Powdery Mildew fungus was conferred by an introgression of genomic DNA of Citrullus lanatus var. citroides on the 2nd chromosome of Citrullus lanatus. WGS analysis have enabled us to learn that the introgression of Citrullus lanatus var. citroides into the Citrullus lanatus genome is between nucleotides 25,000,000-26,200,000.

Preferably, the introgressed sequences are sequenced by way of High Throughput Sequencing (HTS), also termed Next Generation Sequencing (NGS), thereby readily facilitating sequencing the entire genome substantially at once.

Preferably, HTS includes next-generation “short-read” and third-generation “long-read” sequencing methods which applies to exome sequencing, genome sequencing, genome resequencing, transcriptome profiling (RNA-Seq), DNA-protein interactions (ChIP-sequencing), and epigenome characterization.

According to a preferred embodiment, the triploid hybrid watermelon plant comprises a resistance locus genetically linked to at least one marker locus which co-segregates with PM resistance trait and comprises a marker that has been identified for the resistance to PM fungus is conferred by an introgression of genomic DNA of Citrullus lanatus var. Citroides on the chromosome 2 of Citrullus lanatus. Subsequent to WGS analysis, the introgression of Citrullus lanatus var. Citroides into the Citrullus lanatus genome is substantially between nucleotides 24,000,00-25,000,000.

According to a preferred embodiment, the triploid hybrid watermelon plant comprises a resistance locus genetically linked to at least one marker locus which co-segregates with PM resistance trait and comprises a marker that has been identified for the resistance to PM fungus is conferred by an introgression of genomic DNA of Citrullus lanatus var. Citroides on the chromosome 2 of Citrullus lanatus. Subsequent to WGS analysis, the introgression of Citrullus lanatus var. Citroides into the Citrullus lanatus genome is substantially between nucleotides 25,000,000-26,100,000.

According to a preferred embodiment, the triploid hybrid watermelon plant comprises a resistance locus genetically linked to at least one marker locus which co-segregates with PM resistance trait and comprises a marker that has been identified for the resistance to PM fungus is conferred by an introgression of genomic DNA of Citrullus lanatus var. Citroides on the chromosome 2 of Citrullus lanatus. Subsequent to WGS analysis, the introgression of Citrullus lanatus var. Citroides into the Citrullus lanatus genome is substantially between nucleotides 26,100,000-26,200,000.

According to a preferred embodiment, the triploid hybrid watermelon plant comprises a resistance locus genetically linked to at least one marker locus which co-segregates with PM resistance trait and comprises a marker that has been identified for the resistance to PM fungus is conferred by an introgression of genomic DNA of Citrullus lanatus var. Citroides on the chromosome 2 of Citrullus lanatus. Subsequent to WGS analysis, the introgression of Citrullus lanatus var. Citroides into the Citrullus lanatus genome is substantially between nucleotides 26,200,000-28,000,000.

The introgressed sequences are preferably to be found on chromosome 2 in the C. lanatus genome and thus confer resistance to fungi when they are present on chromosomes 2. The introgressed sequences conferring the resistance are more preferably located within the chromosomal region of chromosome 2 which is delimited between nucleotides 24,000,000 and 28,000,000.

The introgressed sequences are preferably to be found on chromosome 2 in the C. lanatus genome and thus confer resistance to fungi when they are present on chromosomes 2. The introgressed sequences conferring the resistance are more preferably located within the chromosomal region of chromosome 2 which is delimited between nucleotides 24,000,000 and 25,000,000.

The introgressed sequences are preferably to be found on chromosome 2 in the C. lanatus genome and thus confer resistance to fungi when they are present on chromosomes 2. The introgressed sequences conferring the resistance are more preferably located within the chromosomal region of chromosome 2 which is delimited between nucleotides 25,000,000 and 26,100,000.

The introgressed sequences are preferably to be found on chromosome 2 in the C. lanatus genome and thus confer resistance to fungi when they are present on chromosomes 2. The introgressed sequences conferring the resistance are more preferably located within the chromosomal region of chromosome 2 which is delimited between nucleotides 26,100,000 and 26,200,000.

The introgressed sequences are preferably to be found on chromosome 2 in the C. lanatus genome and thus confer resistance to fungi when they are present on chromosomes 2. The introgressed sequences conferring the resistance are more preferably located within the chromosomal region of chromosome 2, which is delimited between nucleotides 26,200,000 and 28,000,000.

The present inventors have identified that introgressed sequences essential for the phenotype of interest, i.e. tolerance and/or resistance are to be found in the vicinity of the chromosomal region of chromosome 2 which is delimited between nucleotides 25,900,000 and 26,100,000 or in the vicinity of the chromosomal region of chromosome 2 which is delimited between nucleotides 25,000,000 and 25,900,000, or in the vicinity of both regions. Preferably the introgressed sequences from Citrullus lanatus var. Citroides are to be found in a locus encompassing the position of between nucleotides 26,100,000 and 26,200,000. It is to be noted that the loci of between nucleotides 25,900,000 and 26,100,000 and between nucleotides 26,100,000 and 26,200,000 are located within the chromosomal region defined above.

In some embodiments, the QTL is genetically linked to at least one marker locus that co-segregates with the PM tolerance trait and can be identified by a PCR oligonuclotides primer or a pair of PCR oligonucleotide primers being represented by a forward primer of SEQ ID NO:1 and a reverse primer of SEQ ID NO:2.

The identification of a marker locus identifiable by the pair of PCR oligonucleotide primers represented by the forward primer of SEQ ID NO:1 and the reverse primer of SEQ ID NO:2 is based on System Related Amplified Polymorphism (SRAP) molecular marker system making use of a series of publically available primer pairs for which at least one marker locus of 480 bp has been identified in the genome of all the diploid male seed samples analyzed but was missing from the genome of the female tetraploid.

Specifically, as will be further discussed below, various combinations of the watermelon forward primer sequence identified the commercial name me1 (SEQ ID NO:1), me2, me3, and the reverse primer sequence identified by the commercial name em1 (SEQ ID NO:2), em2, em3, em4 and em5 were used as primer pairs and the combination of me1em1 was positive for a marker locus of 480 bp.

In some embodiment, the genetic background is to be understood as meaning that the allele at the QTL in the cultivated triploid hybrid watermelon genome contributing to the resistance is obtainable from a plant having the genetic background of at least one of PI 482312-PMR or at least one of the cultivated diploid watermelon lines identified herein as PI482312, 572 (Wi1-768-B), or identified herein as 576-a-13-2-SB1 (S12-333-SB1), or identified herein as 573-r-1-2 (PMS12-378-2) or of a progeny or an ancestor of any of these PM tolerant watermelon plants comprising said qualitative trait locus.

Thus, in accordance with the present disclosure there is also provided a triploid hybrid watermelon plant, comprising a resistance locus is genetically linked to at least one marker locus which co-segregates with PM resistance trait and comprises a marker that can be identified in a PCR reaction by amplification of a DNA fragment with a pair of PCR oligonucleotide primers represented by a forward primer of SEQ ID NO:1 and a reverse primer of SEQ ID NO:2 or any other marker located on same chromosome that is statistically correlated and genetically linked to the PM resistance trait.

In some embodiments, the marker locus comprises about 480 base pairs.

In some embodiments, the triploid hybrid watermelon plant disclosed herein is produced by crossing a tetraploid female watermelon with a male plant being a PM tolerant diploid line.

In some embodiments, the male plant selected from the group consisting of lines designated herein W11-701-1; 539 (S09-539-1); 554-F4 (W09-F4-568-1); 554-F5 (BD10-F5-151-13); 554-F7 (S11-F7-548-1); 572 (W11-768-B), 576-a-13-2-SB1 (S12-333-SB1) and 573-r-1-2 (PMS12-378-2).

The present disclosure also concerns seeds of a triploid watermelon plant that produces a watermelon plant characterized by resistance to powdery mildew (PM) and capable of producing seedless fruits with a total soluble solid in the range of 10% to 14%, at times, 11%-13.5% and a red or red-like fruit flesh color.

In some embodiments, the seeds of a triploid watermelon plants are a line designated 512-12.701 having representative seeds deposited on Aug. 24, 2012 under Accession No. NCIMB42044. In yet some other embodiments, the seeds of a triploid watermelon plants are a line designated 12.704bPMI-W12 and having representative seeds deposited on Oct. 14, 2013, under Accession No. NCIMB42172. In yet some other embodiments, the seeds of a triploid watermelon plants are a line designated 12.702b-W14 having representative seeds deposited under Accession No. NCIMB42359. In yet a further embodiment, the seeds of a triploid watermelon plants are a line designated 12.135b-W14 having representative seeds deposited under Accession No. NCIMB42360.

In some embodiments, the seeds of the triploid watermelon plant as disclosed herein are selected as one comprising a resistance locus that is genetically linked to at least one marker locus which co-segregates with PM resistance trait and comprises a marker that can be identified in a PCR reaction by amplification of a DNA fragment with a pair of PCR oligonucleotide primers represented by a forward primer of SEQ ID NO:1 and a reverse primer of SEQ ID NO:2.

Yet, the present disclosure also provides any progeny of a triploid watermelon line disclosed herein. In some embodiments, the progeny has the genetic background of the triploid disclosed herein. In some embodiments, the genetic background is of at least one of the watermelon plants having representative seeds deposited under one of Accession No. NCIMB42044, NCIMB42083, NCIMB42172, NCIMB42359 and NCIMB42360. In yet some other embodiments, the genetic background of the triploid is exhibited in the progeny by at least PM tolerance or by the existence of at least one QTL which contributes to the PM tolerance, or comprises a resistance locus that is genetically linked to at least one marker locus which co-segregates with PM resistance trait and comprises a marker that can be identified in a PCR reaction by amplification of a DNA fragment with a pair of PCR oligonucleotide primers represented by a forward primer of SEQ ID NO:1 and a reverse primer of SEQ ID NO:2.

In some embodiments, the QTL is genetically linked to at least one marker locus that co-segregates with the PM resistance trait and can be identified by a DNA profiling analysis technique, such as, but not limited to, PCR analysis (Polymerase Chain Reaction), SRAP analysis (Sequence-related Amplified Polymorphism) analysis, SSR analysis (Simple Sequence Repeat), AFLP analysis (Amplified Fragment Length Polymorphism) and RAPD analysis (Random Amplified Polymorphic DNA), SNP analysis (Single Nucleotide Polymorphism).

In some embodiments, the triploid hybrid watermelon plant comprises a resistance locus genetically linked to at least one marker locus which co-segregates with PM resistance trait and comprises a marker that has been identified for the resistance to PM fungi is conferred by an introgression of genomic DNA of Citrullus lanatus var. Citroides on the chromosome 2 of Citrullus lanatus. Subsequent to WGS analysis, the introgression of Citrullus lanatus var. Citroides into the Citrullus lanatus genome is substantially between nucleotides 24,000,000-28,000,000.

According to a preferred embodiment, the triploid hybrid watermelon plant comprises a resistance locus genetically linked to at least one marker locus which co-segregates with PM resistance trait and comprises a marker that has been identified for the resistance to PM fungi is conferred by an introgression of genomic DNA of Citrullus lanatus var. Citroides on the chromosome 2 of Citrullus lanatus. Subsequent to WGS analysis, the introgression of Citrullus lanatus var. Citroides into the Citrullus lanatus genome is substantially between nucleotides 24,000,000-25,000,000.

According to a preferred embodiment, the triploid hybrid watermelon plant comprises a resistance locus genetically linked to at least one marker locus which co-segregates with PM resistance trait and comprises a marker that has been identified for the resistance to PM fungi is conferred by an introgression of genomic DNA of Citrullus lanatus var. Citroides on the chromosome 2 of Citrullus lanatus. Subsequent to WGS analysis, the introgression of Citrullus lanatus var. Citroides into the Citrullus lanatus genome is substantially between nucleotides 25,000,000-26,100,000.

According to a preferred embodiment, the triploid hybrid watermelon plant comprises a resistance locus genetically linked to at least one marker locus which co-segregates with PM resistance trait and comprises a marker that has been identified for the resistance to PM fungi is conferred by an introgression of genomic DNA of Citrullus lanatus var. Citroides on the chromosome 2 of Citrullus lanatus. Subsequent to WGS analysis, the introgression of Citrullus lanatus var. Citroides into the Citrullus lanatus genome is substantially between nucleotides 26,100,000-26,200,000.

According to a preferred embodiment, the triploid hybrid watermelon plant comprises a resistance locus genetically linked to at least one marker locus which co-segregates with PM resistance trait and comprises a marker that has been identified for the resistance to PM fungi is conferred by an introgression of genomic DNA of Citrullus lanatus var. Citroides on the chromosome 2 of Citrullus lanatus. Subsequent to WGS analysis, the introgression of Citrullus lanatus var. Citroides into the Citrullus lanatus genome is substantially between nucleotides 26,200,000-28,000,000.

Overall PM—Citrullus lanatus var. Citroides introgression performed is displayed graphically depicting a resistance locus genetically linked to at least one marker locus which co-segregates with PM resistance trait and comprises a marker that has been identified for the resistance to PM fungus is conferred by an introgression of genomic DNA of Citrullus lanatus var. Citroides on the chromosome 2 of Citrullus lanatus. Subsequent to Whole Genome Sequencing (WGS) we performed in 2016 we identified that the resistance to Pordery Mildew fungus was conferred by an introgression of genomic DNA of Citrullus lanatus var. citroides on the 2nd chromosome of Citrullus lanatus. WGS analysis have enabled us to learn that the introgression of Citrullus lanatus var. citroides into the Citrullus lanatus genome is between nucleotides 24,000,000-28,000,000.

The reproduction of triploid progenies may be by any technique known in the art, including, without being limited thereto, cutting, tissue culture, embryo rescue, chromosome doubling, double haploids and mutations. Some methods are described by Wang & You: Study on in-vitro culture of mini watermelon, in Cucurbitaceae, 2012 p. 292-298; Gursoy et al; In vitro ovule and ovarium culture in watermelon, in Cucurbitaceae, 2012 p. 799-804. Wang & You, 2012 and Gursoy et al., 2012 in Cucurbitaceae, 2012]

The present disclosure also provides any seedless watermelon fruit of the triploid watermelon line disclosed herein, the seedless watermelon fruit having at least tolerance to powdery mildew (PM) and one or more of a total soluble solid (TSS) in the range of 10% to 14%; preferably 11%-13.5% and a yellow, orange or red fruit flesh color, preferably dark red.

In some embodiments, the genome of the seedless watermelon fruit of the triploid watermelon line is characterized by genetic background of its male parent having tolerance to resistance to PM.

In some embodiments, the triploid provides a mini fruit with a fruit size in the range of 2 to 4.2 kg. The fruit is typically round.

In some embodiments, the mini fruit are the harvest of a triploid watermelon having representative seeds deposited under the Accession No. NCIMB42044 and/or NCIMB42172 and/or NCIMB42359.

In some other embodiments, the triploid provides a fruit size greater than 4.5 kg, at times greater than 7 kg. The fruit is typically oval.

In some embodiments, the large fruit are the harvest of a triploid watermelon having representative seeds deposited under the Accession No. NCIMB42083 and/or NCIMB42360.

In another aspect, the present disclosure provides a diploid watermelon line having resistance to PM and being capable of pollinating a tetraploid watermelon to produce a triploid hybrid watermelon as disclosed herein.

In general, triploid hybrid watermelon plants are created by crossing a tetraploid female parent line with a diploid male parent line. The tetraploid female parent line and the diploid male parent line are typically planted in a field or greenhouse and the female tetraploid flower is pollinated as known in the art. The triploid seeds that are produced in the fruit of the tetraploid plants are planted in a field with a diploid pollinizer plants to produce fruits that are seedless.

In some embodiments, the diploid watermelon line has a genetic background of the watermelon identified as PI482312 or of P1482312-PMR. The winter 2011 offspring obtained from self-pollination being referred to herein as W11-701-1, at least with respect to the trait of resistance to PM, as well as any diploid PM tolerant plant selected from the lines designated herein 572 (W11-768-B), or designated herein 576-a-13-2-SB1 (S12-333-SB1), or designated 573-r-1-2 (PMS12-378-2).

In some embodiments, the diploid watermelon line disclosed herein may be characterized by lines referred to herein as 539 (S09-539-1), 554-F5 (BD10-F5-151-13) and 554-F7 (S11-F7-548-1) all having the genetic background of at least one of the disclosed and deposited male parent diploid plant, and/or at least comprising a resistance locus being linked to a genetic background or genetic determinant obtainable from the genome of a wild watermelon plant having resistance to PM, such as the PM watermelon plant designated according to the plant index PI 482312 or of the cultivated and herein deposited diploid watermelon plants.

In some embodiments, the diploid watermelon line disclosed herein has resistance to one or both of PMW1 and PMW2.

In accordance with this aspect, there is also provided pollen of a diploid watermelon line disclosed herein.

Yet further disclosed herein are the seeds of the said diploid watermelon line as well as any other form for obtaining progeny of the diploid watermelon. Thus in the context of the present disclosure, when referring to diploid watermelon line having resistance to one or both of PMW1 and PMW2 it is to be understood as encompassing also any sexually or asexually reproduced progeny of the above recited diploid male watermelon plant lines.

The diploid watermelon line disclosed herein may be further characterized independently by any one of the following parameters. Therefore, while being presented herein as a list, each parameter below constitutes an independent embodiment of the present disclosure and may be combined with one or more other parameter from this list:
vigor of between 60 and 90, typically about 70;
earliness of between 25 and 50, typically about 30-40;
flower setting of between 30 to 50, at times 30-40;
flower sex being monoecious (M).
rind color of tiger (narrow stripes) or gray or crimson (wide dark stripes); preferably gray.
number of fruits per plant in the range of 2 to 4.
fruit regularity in the range of 60 to 80;
fruit weight of between 2 to 12 kg.
flesh color being yellow, orange or red fruit flesh color, preferably dark red.
flesh firmness between 60 to 80, typically about 70.
rind thickness between 9 mm to 11 mm, typically about 10 mm.
rind crack of between 10 to 80, typically about 10.
seed color being brown or black.
weight of a 1,000 seeds being between 20 to 65.
seed size of between 4-6×6-11 mm.
TSS of at least 10%, at times between 10% and 13%;
PMW1 and PMW2 resistance in the range of 6-8 (also referred to as intermediate resistance).

In accordance with another aspect, the present disclosure also provides a tetraploid female watermelon selected from the line designated “367-1”, including any sexually or asexually produced progeny thereof having a flesh color selected from yellow, orange, red, dark red or red like, and TSS of at least 10%.

To create tetraploid female watermelon line, it is known in the art to use chemicals that affect mitotic processes of a diploid inbred line so as to affect the number of chromosomes that are eventually obtained. The best known chemical is colchicine. Oryzaline, ethalfluralin and dinitroanilines have also been used for doubling of existing chromosome content.

The diploid line used to create a tetraploid is selected based on the desired traits for the tetraploid line. Methods for developing tetraploid plants are described in the art [Kihara H. 1951, triploid watermelons. Processings of American Society of Horticultural Science 58:217-230; Eigstri O. J., 1971 Seedless Triploids. HortScience 6:1-2; Donald N. Maynard, Watermelons, characteristics, Production and Marketing, pp. 63-67, 2001]

Cross pollination between a tetraploid line, the female parental line, and a diploid line, the male parental line, are accomplished by either hand or insects (mostly bees) pollination. In bee pollination all the male flowers covered by capsules and the female flowers are open pollinated by bees. In hand pollination Male flowers of the diploid male parent line are collected in the early morning before the visit of insects and female flower buds are uncovered where pollination with the collected fresh male flowers is performed, e.g. by hand.

The tetraploid watermelon plant may be characterized by one or more of the following parameters. Therefore, while being presented herein as a list, each parameter below constitutes an independent embodiment of the present disclosure and may be combined with one or more other parameter from this list:
vigor of between 40 and 70;
earliness of between 50 and 70;
flower setting of between 50 to 70;
flower sex being monoecious (M).
rind color crimson (wide dark stripes) or tiger (narrow dark stripes) or gray, preferably crimson;
number of fruits per plant in the range of 2 to 4.
fruit regularity in the range of 60 to 80;
fruit weight of between 2 to 8.5 kg.
flesh color being yellow, orange or red fruit flesh color, to deep/dark red.
flesh firmness between 60 to 80, typically about 70.
rind thickness between 12 mm to 16 mm.
rind crack of between 10 to 80, typically about 10.
seed color being brown or black.
weight of a 1,000 seeds (TSW) being between 25 g. to 90 g.,
seed size of between 4-7×7-12 mm.
TSS of at least 10%, at times between 10% and 13%
PMW1 and PMW2 sensitive (in average scaled 2).

In yet a further aspect, there is provided by the present disclosure a method of producing a triploid hybrid watermelon plant characterized by resistance to powdery mildew (PM) and capable of producing seedless fruits with a total soluble solid (TSS) in the range of 10% to 14%, and a yellow, orange or red fruit flesh color, preferably dark red fruit flesh color, the method comprising:
a. planting a field with a PM sensitive female parent tetraploid watermelon line having a total soluble solid of at least 10%, and yellow, orange or red fruit flesh color, preferably dark red fruit flesh color, flesh firmness of between 60 to 80 and TSW of between 25 g. and 90 g.,
b. pollinating said tetraploid watermelon line with pollen from a male watermelon line having tolerance to PM, to produce triploid watermelon seeds;
c. planting said triploid watermelon seeds to produce said triploid watermelon plants having resistance to PM.

Further provided is a method of producing a triploid hybrid watermelon fruit harvested from a triploid watermelon plant with tolerance to powdery mildew (PM) and the fruit being seedless, and with a total soluble solid in the range of 10% to 14%, preferably in the range of 11% to 13.5% or about 13.5%, and red fruit flesh color, the method comprising:
a. planting a PM sensitive female parent tetraploid watermelon line having a total soluble solid of at least 10%, yellow, orange or red fruit flesh color, preferably dark red fruit flesh color, flesh firmness of 80 and TSW of between 25 g. to 90 g.,
b. pollinating said tetraploid watermelon line with pollen from a male parent diploid watermelon line having tolerance to PM, to produce triploid watermelon seeds;
c. planting said triploid hybrid watermelon seeds and exposing the triploid hybrid plant to a pollinizer to produce said triploid watermelon plants with seedless fruits.
d. harvesting triploid seedless watermelon fruit from said triploid hybrid watermelon plants.

In some embodiments, the male watermelon is selected from the group consisting of lines designated 539 (S09-539-1); 554-F4 (W09-F4-568-1); 554-F5 (BD10-F5-151-13); 554-F7 (S11-F7-548-1), 572 (W11-768-B), 576-a-13-2-SB1 (PMS12-333-SB1), and 573-r-1-2 (PMS12-378-2).

In some embodiments, the male parent diploid watermelon line is selected by identifying a marker locus which co-segregates with PM resistance trait using, said identification comprises a PCR reaction comprising amplification of a DNA fragment with a pair of PCR oligonucleotide primers represented by a forward primer of SEQ ID NO:1 and a reverse primer of SEQ ID NO:2 or any other marker located on same chromosome that is statistically correlated and genetically linked to the PM resistance trait.

In some embodiments, the male diploid is identified when said marker comprises or has about 480 base pairs.

In some embodiments, planting is of only triploid watermelon seeds having a genetic background of the selected male parent.

In some embodiments, the planting is of seeds that their genetic background comprises a marker locus identifiable by a PCR reaction comprising amplification of a DNA fragment with a pair of PCR oligonucleotide primers represented by a forward primer of SEQ ID NO:1 and a reverse primer of SEQ ID NO:2.

In some embodiments, the method comprises selecting triploid watermelon seeds comprising in their genome a PM resistance locus that is genetically linked to at least one marker locus which co-segregates with the PM resistance trait, said selection comprises identifying said marker with a PCR amplification reaction of a DNA fragment with a pair of PCR oligonucleotide primers represented by a forward primer of SEQ ID NO:1 and a reverse primer of SEQ ID NO:2 and planting triploid watermelon seeds for which said marker has been identified.

After the triploid is planted, the plants are allowed to grow until harvesting. In some embodiments, the harvesting is about 35 to about 45 days after flowering.

As indicated above, pollination may be insect pollination or hand pollination. In accordance with some embodiments, the pollination is hand pollination. Pollination occurs in anthesis (anthesis is the period during which a flower is fully open or sexually functional). The male flowers are collected and the pollen spread on the female flower stigma by hand. In some other embodiments, pollination is by insects (mostly bees).

In some embodiments, the method requires emasculating the female parent tetraploid watermelon prior to pollination and the triploid hybrid watermelon plant prior to pollination.

In the description above and below the watermelon plant is referred to either by an internal identification code or by a deposit Accession No. For each of reference, the following provides a correlation between the internal reference/name and the NCIMB deposit Accession No.

| | Internal reference/name | Accession No. | Date of deposit |
|---|---|---|---|
| Triploid | S12-12.701 | NCIMB42044 | 24 Aug. 2012 |
| Triploid | 12.704bPMI-W12 | NCIMB42172 | Oct. 14, 2013 |
| Triploid | 12.702b-W14 | NCIMB42359 | Feb. 16, 2015 |
| Triploid | 12.135b-W14 | NCIMB42360 | Feb. 16, 2015 |

### DESCRIPTION OF SOME NON-LIMITING EXAMPLES

### Example 1: Production of Mini Seedless Triploid Watermelons

The target of this breeding was to develop mini seedless watermelon hybrids tolerant to PM, using the genetic background of PI 482312. Unless otherwise stated, all breeding stages were conducted in plastic greenhouses in Bnei Darom, Israel.

### The Male Parent Lines

PI 482312 is a watermelon (Citrullus lanatus var. Citroides) line known to have tolerance to powdery mildew (PM). This PI was obtained from the plant genetic resources unit, Griffin, Georgia, originally from Zimbabwe. PI 482312 exhibited heterogeneity in PM tolerance. Therefore, selections of self-pollination of this PI line was conducted to obtain a line homogeneous for high level of PM resistance (i.e. in a scale of between 1 to 9, to a level of 8-9), and this line is referred to herein as PI 482312-PMR. To this end, the plants of PI 482312-PMR were routinely grown in plastic greenhouse during the years 2005-2012, in two seasons per year, winter and summer. During the routine growth, observations (including microscopic) revealed that resistance of the self-pollinated plant showed no symptoms or very minor symptoms.

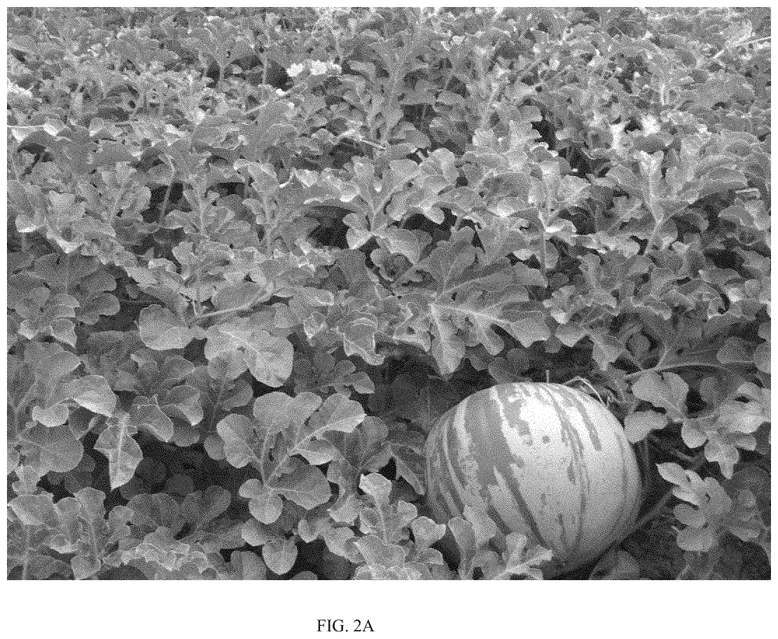

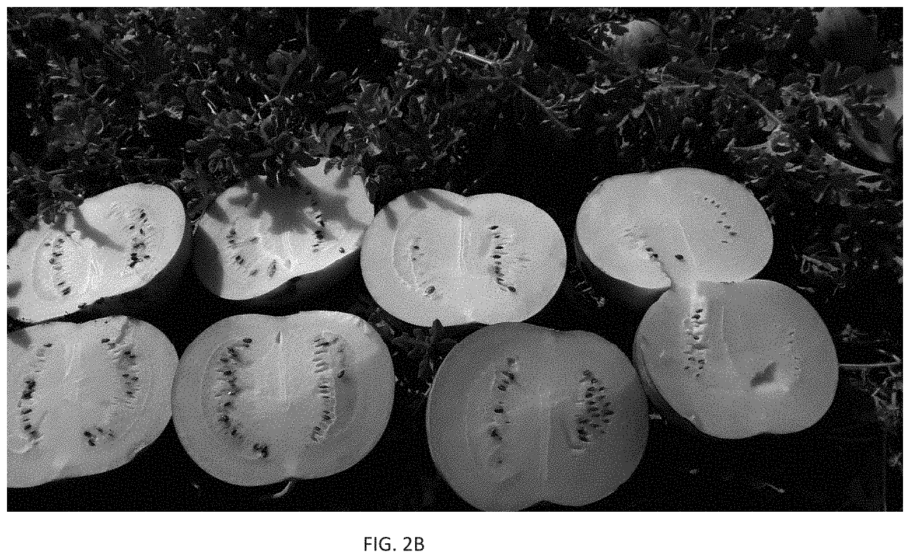

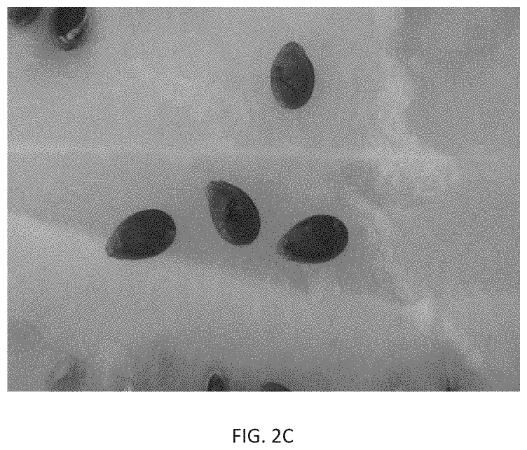

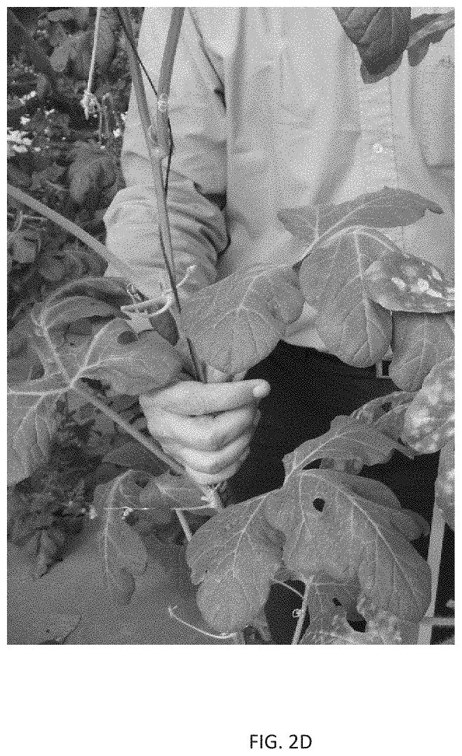

The original plant, PI 482312-PMR, is also characterized by a large fruit of about 10 kg, having a gray rind with non-continuous (“broken”) green stripes (FIG. 2A), white to yellow fruit flesh (FIG. 2B) and non-bitter taste, Total soluble solids (TSS) of 6% in average. The seeds color is light green and big (about 8 mm*13 mm) (FIG. 2C). PI 482312-PMR plants are PM tolerate, ranked 9, (FIG. 2D).

### Development Stages (Year Description):

The target of this breeding was to develop seedless watermelon hybrids resistance to PM, using the genetic background of PI 482312-PMR. Unless otherwise stated, all breeding stages were conducted in plastic greenhouses in the southern areas of Israel.

In the following the breeding lines were exposed to PM where those showing best tolerance were selected. Specifically, the plants were grown in greenhouse and were inoculated by spreading uniformly the fungus by hand in the greenhouse. Also, the plants in the greenhouse were not treated with fungicides. In a scale from 1 to 9 (9 being the best performance of PM resistance), the male parent lines exhibited resistance to PMW1 of 7-8. The male parent lines exhibited resistance to PMW2 of 7-8.

The breeding pathways which are also illustrated in Tables 1A-1C below are as follows.

### Pathway A (403, 433 &539 PM Tolerance Selection):

Summer 2005: a first cross between PI 482312-PMR and diploid inbred watermelon line “CLS” (selection from Cal sweet variety), as a cultured diploid line, created line “403” (PI×CLS). Each cross and breeding line were exposed to PM and the most tolerant plants were selected for further breeding. The plants were grown in greenhouse and were inoculated by spreading uniformly the fungus by hand in the greenhouse. Also, the plants in the greenhouse were not treated with fungicides.

Winter 2005: a backcross between line “403” and a mini diploid inbred watermelon line “126” (proprietary of Origene Seeds Ltd.) created line “433” (403×126).

Summer and winter 2006: the first generation cross of line “433” namely “433-F1” was self-pollinated two generations to provide F3 generation, namely, “433-F3”.

Summer 2007: a backcross between “433-F3” and a mini diploid inbred watermelon line “112” (proprietary of Origene seeds Ltd.) create line “539”.

Winter 2007-Summer 2009: the first generation of “539”, namely, “539-F1” was promoted four generations to provide F5 generation, namely, “539-F5” (hereinafter, referred to as “S09-F5-539-1” for Summer 09, 5^{th} generation of 539).

Winter 2009: a cross between S09-F5-539-1 and a tetraploid female line “367-1” (proprietary of Origene seeds Ltd.) created a first generation, F1, of seedless hybrid tolerant to PM. This F1 generation was referred to as “W09-9701”.

### Pathway B (406&554 PM Tolerance Selection):

Summer 2005-Winter 2007: two diploid lines “237” and “406” were promoted separately five generations, with “406” being PM resistant (PMR) and “237” being PM sensitive (PMS). Both lines are diploid inbred watermelon, both a proprietary Origene. The offspring of the five generation, i.e. “237-F6” and “406-F6” were obtained in Winter of 2007.

Each cross and breeding line were exposed to PM and the most tolerant plants were selected for further breeding.

Summer 2008-Summer 2011: a cross between “237-F6” and “406-F6” took place, to create a new diploid male line “554-F1”. The “554-F1” was then promoted 3, 4, 5, 6 and 7 generations to provide, respectively, the plants “554-F4” (also referred to as “W09-F4-568-1”), “554-F5” (also referred to as BD10-F5-151-13), “554-F6” (also referred to as W10-F6-642-1) and “554-F7” (also referred to as S11-F7-548-1).

Winter 2009: Crossing between the tetraploid female line “367-1” and the diploid male line “554-F4” to obtain in the summer of 2010 an improved seedless hybrid tolerant to PM, which was referred to as “S10-9701i”, the improvement being in terms of better tolerance to PM as compared to male lines “S09-F5-539-1”.

Summer 2010: Crossing between the tetraploid female line “367-1” and the diploid male line “554-F5” to obtain in the Winter of 2010 an improved seedless hybrid tolerant to PM which is referred to as “W10-9701i=10.720”, the improvement being in terms of better tolerance to PM as compared to male lines “554-F4” to “554-F5”.

Summer 2011: Crossing between the tetraploid female line “367-1” and the diploid male line “554-F7” (also referred to as “S11-F7-548-1”) to obtain in the Winter of 2011 an improved seedless hybrid tolerant to PM which is referred to as “W11-10.720”, the improvement being in terms of better tolerance to PM as compared to male lines “554-F5” to “554-F7”.

### Pathway C (572 PM Tolerance Selection):

With the aim to improve the quality of the diploid male line “554-F5” (“BD10-F5-151-13”) with horticultural traits such as fruit regularity, smaller fruit and deeper red flesh, a third breeding pathway was employed.

Summer 2010: Backcross of “554-F5” with a diploid inbred watermelon line “112” (proprietary line of Origene Seeds Ltd.) to create male line “572-F1”.

Winter 2010-Winter 2011: The line “572-F1” was promoted three generations to “572” (also referred to as “W11-768-B”).

Each cross and breeding line were exposed to PM and the most tolerant plants were selected for further breeding.

Winter 2011: Crossing between male line “572” (this line was marked under the name “W11-768-B”) and the tetraploid female line “367-1” to obtain in the Summer of 2012 an improved F1 seedless hybrid referred to as “S12-12.701”

TABLE 1A
Breeding pathway A

| Season, | Female | Male | | Male PM |
|---|---|---|---|---|
| year | (♀) | (♂) | Hybrid (F1) | selection |
| Summer, 2005 | | 403-F1 | | + |
| | | (PI* × CLS) | | |
| Winter, 2005 | | 433-F1 | | + |
| | | (403 × 126) | | |
| Summer, 2006 | | 433-F2 | | + |
| Winter, 2006 | | 433-F3 | | + |
| Summer, 2007 | | 539-F1 | | + |
| | | (433 × 112) | | |
| Winter, 2007 | | 539-F2 | | + |
| Summer, 2008 | | 539-F3 | | + |
| Winter, 2008 | | 539-F4 | | + |
| Summer, 2009 | 367-1 | 539-F5 | | + |
| | | (S09-F5-539-1) | | |
| Winter, 2009 | | | W09-9701 | |

*PI denotes PI 482312-PMR

TABLE 1B
Breeding pathway B

| Season, | Female | Male | | Male PM |
|---|---|---|---|---|
| year | (♀) | (♂) | Hybrid (F1) | selection |
| Summer, 2005 | | 237-F1, 406-F1 | | + |
| | | (601F × PI*) | | |
| Winter, 2005 | | 237-F2, 406-F2 | | + |
| Summer, 2006 | | 237-F3, 406-F3 | | + |
| Winter, 2006 | | 237-F4, 406-F4 | | + |
| Summer, 2007 | | 237-F5, 406-F5 | | + |
| Winter, 2007 | | 237-F6, 406-F6 | | + |
| Summer, 2008 | | 554-F1 (237-F6 × | | + |
| | | 406-F6) | | |
| Winter, 2008 | | 554-F2 | | + |
| Summer, 2009 | | 554-F3 | | + |
| Winter, 2009 | 367-1 | 554-F4 (W09-F4- | | + |
| | | 568-1) | | |
| Summer, 2010 | 367-1 | 554-F5 (BD10-F5- | S10-9701i | + |
| | | 151-13) | | |
| Winter, 2010 | | 554-F6 (W10-F6- | W10-9701i = | + |
| | | 642-1) | 10.720 | |
| Summer, 2011 | 367-1 | 554-F7 (S11-F7- | | + |
| | | 548-1) | | |
| Winter, 2011 | | | W11-10.720 | |

*PI denotes PI 482312-PMR

TABLE 1C
Breeding pathway C

| Season, | Female | Male | | Male PM |
|---|---|---|---|---|
| year | (♀) | (♂) | Hybrid (F1) | selection |
| Summer, 2010 | | 572-F1 | | + |
| | | (554-F5 × 112) | | |
| Winter, 2010 | | 572-F2 | | + |
| | | (W10-F2-646-1) | | |
| Summer, 2011 | | 572-F3 | | + |
| | | (S11-F3-553-d) | | |
| Winter, 2011 | 367-1 | 572-F4 | | + |
| | | (W11-F4-768-B) | | |
| Summer, 2012 | | | S12-12.701 | |

### Characterization of Male Parent Lines:

The male parent lines were characterized by the following parameters:

Fruit:
round;
gray rind color (at times showing thin, light green stripes) with thickness of 10 mm in average;
   red to deep yellow, orange or red fruit flesh color, preferably dark red flesh;
   rind firmness of 10, namely, with low tendency of cracking;
   good taste (total soluble solids being in average 11%)

Seeds:
brown color;

PM Tolerance to Resistance:

In a scale from 1 to 9 (9 being the best performance of PM resistance), the four male parent lines exhibited resistance to PMW1 of 7-8, and male parent lines exhibited resistance to PMW2 of 7-8.

Flowering:

The plants of all four lines flower in the early season. The flowers are monoecious. In a scale from 1 to 90 (90 being the best performance), the four parent males lines exhibited strong plant vigor of 70.

In overall and as evident from Table 2, all male lines commonly share the same characteristics, but still differ in some, including fruit size (weight), flesh color and seed size (Thousand Seeds Weight, TSW). Some differences also appeared in the level of tolerance to PM.

TABLE 2
Page 27: Characteristics of male lines and the selected female 367-1:

| Line | PI 482312-PMR | 539-F5 | 554-F5 | 554-F7 | 572-F4 | 367-1 |
|---|---|---|---|---|---|---|
| Characteristics | W11-F99-701-1¹ | (S09-F5-539-1) | (BD10-F5-151-13) | (S11-F7-548-1) | (W11-F4-768-B) | (W11-F99-448-B11) |
| Vigor | 90 | 70 | 70 | 70 | 70 | 45 |
| Earliness | 90 | 40 | 40 | 40 | 40 | 50 |
| Setting | 20 | 50 | 30 | 30 | 30 | 70 |
| Flower sex | M | M | M | M | M | M |
| Rind color | Citron | Gray | Gray | Gray | Gray | Crimson dark |
| No. fruit/plant⁶ | 2 | 3 | 3 | 3 | 3 | 3 |
| Fruit shape | Flat round | Round | Round | Round | Round | Round |
| Fruit regularity | 60 | 60 | 60 | 70 | 80 | 80 |
| Fruit striping | 20%² | 10%³ | 10%³ | 10%³ | 10%³ | 80%⁴ |
| Fruit weight (Kg) | 10 | 4 | 8 | 8 | 4 | 3 |
| Flesh color | White to yellow | Red | Red | Red | Deep red | Deep red |
| Flesh firmness | 90 | 70 | 70 | 70 | 70 | 70 |
| Rind thickness | 15 | 10 | 10 | 10 | 10 | 12 |
| (mm) | | | | | | |
| Rind Crack | 10 | 10 | 10 | 10 | 10 | 10 |
| Seed color | Light green | Brown | Brown | Brown | Brown | Brown |
| TSW⁵ (gr) | 180 | 24 | 28 | 44 | 48 | 60 |
| Seed size (mm) | 8*13 | 4.5*6.5 (oval) | 4*7 | 5.5*8.5 (oval) | 5.5*8.5 (oval) | 6*9 |
| Taste | Not bitter | Good | Good | Good | Good | Good |
| TSS (Brix %) | 6 | 11 | 11 | 11 | 11 | 11 |
| PMW1 | 9 | 8 | 7 | 8 | 8 | 2 |
| PMW2 | 9 | 2 | 7 | 8 | 8 | 2 |

¹F99 is a symbol for commercial/end of breeding line
²20% indicating Gray rind with incomplete (broken) green stripes that cover about 20% of the fruit's rind;
³10% indicating gentle thin (pencil-like) light green stripes;
⁴80% indicating Gray rind with wide dark green stripes that cover about 80% of the fruit’s rind;
⁵Weight of 1,000 seeds;
⁶harvesting 35-45 days after pollination

### Description of the Female Parent

The tetraploid female parent line was named “367-1” (this line was marked under the name S4W11-F99-448-B, and was obtained from a cross between two tetraploids referenced as lines “326” and “330” (both proprietary tetraploid lines of Origene Seeds Ltd.). Upon PM tolerance testing, the female line “367-1” was ranked 2 in terms of sensitivity to PM.

The characteristics of “367-1” female line are also provided in Table 2. Generally, the female line produces round shape fruits of an average weight of 3 kg with dark crimson rind color. The rind thickness is 12 mm. The fruits flesh is yellow, orange or red fruit flesh color, preferably dark red with 11% TSS. The seeds color is brown, and TSW is 60 g. The plants flower in the middle season.

### Description of the Tolerant Seedless Hybrids

Plants of the male parent lines 539 (S09-539-1), 554-F4 (W09-F4-568-1), 554-F5 (BD10-F5-151-13), 554-F7 (S11-F7-548-1), and 572 (W11-768-B) were crossed with the tetraploid female line 367-1 (resulting in a series of PM-tolerant, seedless hybrids named W09-9701, S10-9701i, W10-9701i (10.720), W11-10.720 and S12-12.701.

The characteristics of the hybrid lines are provided in Table 3.

In general, unless otherwise stated, the plants were grown in a plastic greenhouse in Bnei Darom, Israel. Hand pollination took place, and included collection of males flowers from the male diploid plants and brushing the flowers over the flowers on the female tetraploid plants, in the early morning (7:00-11:00 AM).

The F1 generation from crossing between 539 (S09-539-1) and 367-1 (i.e. 539x367-1) resulted in the W09-9701 hybrid. The W09-9701 hybrid was initially tested for its tolerance to PM, by artificial (hand spreading) inoculation. Tolerance was tested to both PMW1 in early season and PMW2 in late season.

As shown in Table 3, the “W09-9701” hybrid was found to have a high degree of tolerance to PMW1 (ranked 6) but some sensitivity to PMW2 (ranked 2). Namely, the “W09-9701” hybrid was more tolerant to PMW1 than to PMW2.

Improvement of fruit quality (deeper red flesh color and better fruit regularity) was also required. Therefore, an additional crossing took place. Specifically, the hybrids S10-9701i and W10-9701i (10.720) were produced as a result of pollination between the male parent line 554-F4 (W09-F4-568-1) and 554-F5 (BD10-F5-151-13) and the female parent 367-1 namely to produce the hybrids of (554-F4x367-1) and (554-F5x367-1).

The W10-9701i hybrid was tested in an open field in Israel and was also found to have tolerance to both PMW1 and PMW2 (ranking 6 for both pathogens). In addition, the W10-9701i hybrid had an exceptional fruit quality in terms of flesh color (deeper red) and a higher total soluble solids percentage in the fruit (11.5%).

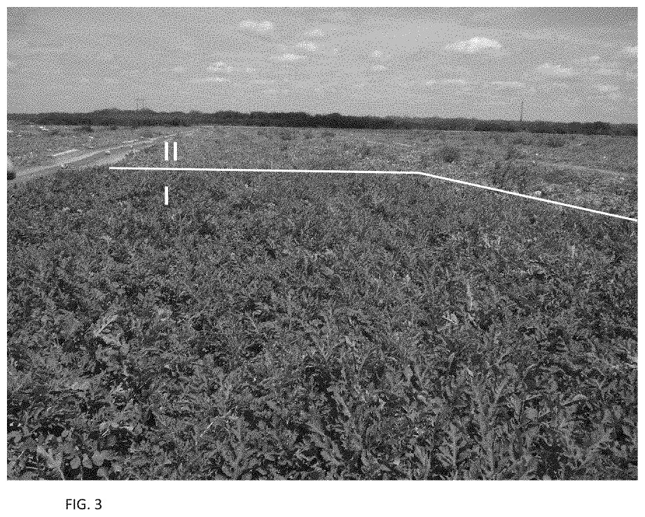

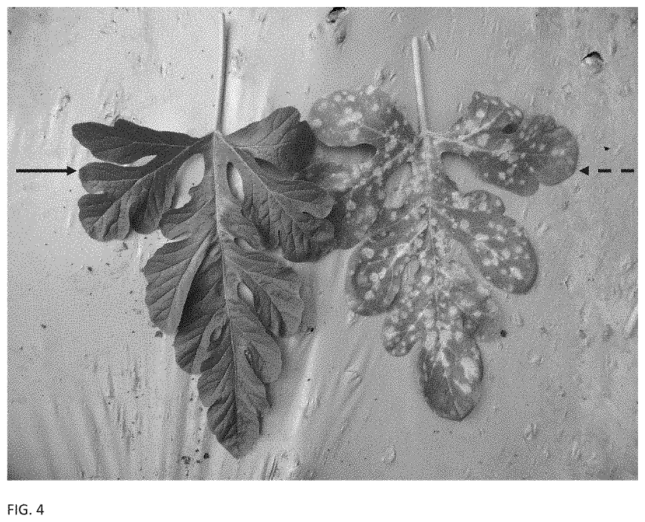

The tolerance of the W10-9701i hybrid was also tested in an open field in Brazil, and was found to have high tolerance to both strains of PM (PMW1 and PMW2) as compared to a reference seedless watermelon hybrids (known by the commercial name Leopard (Hazera Israel) tested in the same field, as shown in FIG. 3. Further, FIG. 4 clearly shows that the tolerant watermelon plant after inoculation lacks white mycelium and white spores on both sides of the leaves (full arrow) which are evident on the reference plant (dashed arrow).

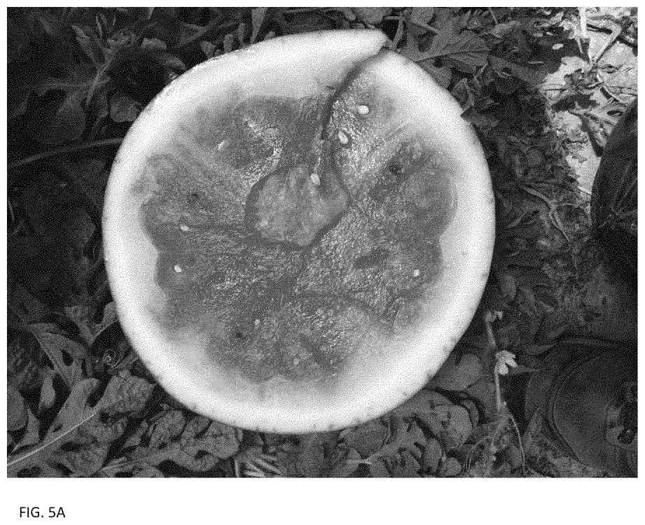

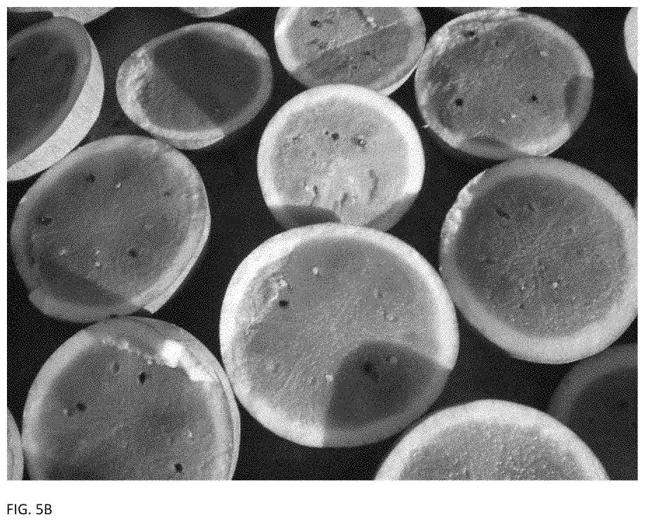

Additional hybrids were produced by hand pollination between the male parent line 554-F7 (S11-F7-548-1) and the same female parent 367-1 (554-F7×367-1) to produce the hybrid W11-10.721. Also, crossing was between male parent line 572 (W11-768-B) and again the female parent 367-1 (572-F4×367-1) to produce the hybrid S12-12.701. In this connection, FIGS. 5A and 5B show fruit quality, in particular fruit rind thickness and flesh color uniformity of a fruit at intermediate stage of development (FIG. 5A, corresponding to male line 554 in terms of flesh color uniformity), and of a later line, from which the triploid seedless watermelon of the invention may be produced (FIG. 5B, corresponding to male line W11-768-B, in terms of flesh color uniformity). Specifically, FIG. 5B shows the rind being thin and the flesh being uniform.

As shown in Table 3 all the hybrid lines had a dark crimson rind color and the fruits were essentially round. The weight of the fruits was between 3 to 4.5 Kg and all hybrids showed high tolerance to PMW1, the tolerance ranged 6-7 out of 9.

TABLE 3
Characteristics of the triploid hybrid lines

| Hybrid line | W09-9701 | S10-9701i | W10-9701i (10.720) | W11-10.720 | S12-12.701 |
|---|---|---|---|---|---|
| Vigor | 50 | 50 | 50 | 50 | 50 |
| Earliness | 50 | 50 | 50 | 45 | 45 |
| Setting | 50 | 50 | 50 | 50 | |
| Flower sex | M | M | M | M | M |
| Rind color | Dark crimson | Dark crimson | Dark crimson | Dark crimson | Dark crimson |
| No. fruit/plant | 2 | 2 | 2 | 2 | 2 |
| Fruit shape | Round | Round | Round | Round | Round |
| Fruit regularity | 65 | 65 | 65 | 70 | 80 |
| Fruit striping | 80%¹ | 80%¹ | 80%¹ | 80%¹ | 80%¹ |
| Fruit weight (Kg) | 3.3 | 4.5 | 4.5 | 4.5 | 3.2 |
| Flesh color | Light red | Light red | Red | Red | Deep red |
| Flesh firmness | 70 | 70 | 70 | 70 | 70 |
| Rind thickness (mm) | 12 | 12 | 12 | 12 | 12 |
| Rind Crack | 10 | 10 | 10 | 10 | 10 |
| Seed color | Seedless | Seedless | Seedless | Seedless | Seedless |
| Taste | Good | Good | Good | Good | Very good |
| TSS (Brix %) | 11 | 11.5 | 11.5 | 11.5 | Not tested yet |
| PMW1 | 6 | 6 | 6 | 7 | 7 |
| PMW2 | 2 | 6 | 6 | 7 | 7 |

¹Gray fruit with wide dark green stripes that cover 80% of the fruit

### Example 2: Production of Triploid Mini Seedless Watermelon with PM Resistance—Development Stages (Year Description)

The target of this breeding was to develop seedless watermelon hybrids tolerant to PM, using the genetic background of PI 482312 as described above. Unless otherwise stated, all breeding stages were conducted in plastic greenhouses in the southern areas of Israel. Breeding stages were similar to those described in Example 1 above.

The following breeding lines were exposed to PM and those showing best tolerance were selected (by scoring, see below). Specifically, the plants were grown in a greenhouse and were inoculated by spreading uniformly the fungus by hand in the greenhouse. The plants in the greenhouse were not treated with fungicides at all. In a scale from 1 to 9 (9 being the best performance of PM resistance), the male parent line exhibited resistance to PMW1 of 7-8 and male parent line exhibited resistance to PMW2 of 7-8.

The breeding pathway is also illustrated in the following Table 4 below.

12.704bPMI (Classico2 Type PMI*) Pathway (Males 406, 554 & 573 PM Tolerance Selection):

Male:

Summer 2005: a first cross between PI 482312 and diploid inbred watermelon line “601F” (proprietary of Origene seeds Ltd.), created the line “406-F1” (601F×PI).

Winter 2005 till winter 2007: The line “406-F1” was self-pollinated five generations to provide F6 generation, namely “406-F6”.

Each generation was exposed to PM and the most tolerant plants were selected for further breeding. The plants were grown in greenhouse and were inoculated by spreading uniformly the fungus by hand in the greenhouse. The plants in the greenhouse were not treated with fungicides at all. In parallel each generation was selected for big fruit size being greater than 7 Kg (about 10-14 kg) and quality flesh traits including uniform flesh color of yellow, orange or red fruit flesh color, preferably dark red, TSS between 11%-12%.

Summer 2008: a backcross between “406-F6” and a small (2-3 Kg) diploid inbred watermelon line “237-F6” (proprietary of Origene seeds Ltd.) created the line “554-F1”.

Winter 2008 till summer 2010: The line “554-F1” was self-pollinated four generations to provide F5 generation, named “554-b-1-1” (“554-F5”). The plants were grown in greenhouse and were inoculated by spreading uniformly the fungus (by hand) in the greenhouse. The plants in the greenhouse were not treated with fungicides at all.

Summer 2010: Another backcross between “554-F5” and a small (1-2 Kg) diploid inbred watermelon line “216-a-2-B-5-B” (“216-F11”) (proprietary of Origene seeds Ltd.) created the line “573-F1”.

Summer 2010 till summer 2013: The line “573-F1” was self-pollinated seven generations to provide F7 generation, namely “573-r-1-2-1-B” (PMS13-642-B1). Representative generation F5 (PMS12-F5-378-2).

Each cross and breeding line were exposed to PM and the most tolerant plants were selected for further breeding. The plants were grown in greenhouse and were inoculated by spreading uniformly the fungus by hand in the greenhouse. The plants in the greenhouse were not treated with fungicides at all.

Triploid:

Winter 2012: a cross between PMS12-F5-378-2 and a tetraploid female line “SS367-1” (proprietary of Origene seeds Ltd. created a first generation, F1, of seedless hybrid tolerant to PM. This F1 generation was referred to as “12.704bPMI-W12” (“Classico2 type PMI”, PMI indicating tolerance of 6-7). This F1 generation of the seedless tolerant triploid hybrid watermelon was deposited on Oct. 14, 2013, under Accession No. NCIMB42172.

TABLE 6
12.704b PMI (Classico type PMI) Breeding

| Season, | Female | Male | | Male PM |
|---|---|---|---|---|
| year | (♀) | (♂) | Hybrid (F1) | selection |
| Summer, 2005 | | 406-F1 (601F × | | + |
| | | PI*) | | |
| Winter, 2005 | | 406-F2 | | + |
| Summer, 2006 | | 406-F3 | | + |
| Winter, 2006 | | 406-F4 | | + |
| Summer, 2007 | | 406-F5 | | + |
| Winter, 2007 | | 406-F6 | | + |
| Summer, 2008 | | 554-F1 (406 × | | + |
| | | 237-F6) | | |
| Winter, 2008 | | 554-F2 (W08-F2- | | + |
| | | 554-B) | | |
| Summer, 2009 | | 554-F3 (554-b, S09- | | |
| | | F3-280-2) | | |
| Winter, 2009 | | 554-F4, 554-b-1, | | + |
| | | W09-F4-568-1) | | |
| Summer, 2010 | | 554-F5 (554-b-1-1 | | + |
| | | S10-F5-151-1) | | |
| | | 573-F1 (554-F5 × | | |
| | | 2216-F11) | | |
| Winter, 2010 | | 573-F2 (W10-F2- | | + |
| | | 647-B) | | |
| Summer, 2011 | | 573-F3 (573-r, | | + |
| | | BD11-F3-555-r) | | |
| Winter, 2011 | | 573-F4 (573-r-1, | | + |
| | | PMW11-F4-782-1) | | |
| Summer, 2012 | | 573-F5 (573-r-1-2, | | + |
| | | PMS12-F5-378-2) | | |
| Winter, 2012 | 367-1 | 573-F6 (573-r-1-2- | | + |
| | | 1, PMW12-F6-625- | | |
| | | 1) | | |
| | 367-1 | 573-F5 (573-r-1-2 | 12.704bPMI- | |
| | | PMS12-F5-378-2) | W12 | |
| Summer, 2013 | | 573-F7 (573-r-1-2- | | + |
| | | 1-B1 PMS13-F7- | | |
| | | 642-B1) | | |
| Winter, 2013 | 367-1 | 573-F7 (573-r-1-2- | 12.704bPMI- | |
| | | 1-B1 PMS13-F7- | W13 | |
| | | 642-B1) | | |

*PI denotes PI-482312-PMR

TABLE 7
Characteristics of male line “573-r-1-2”, female line 367-1
and first generation hybrid 12.704bPMI-W12 (NCIMB42172):

| Line | 573-r-1-2 | | |
|---|---|---|---|
| characteristics | PMS12-F5-378-2 | 367-1 | 12.704b |
| Vigor | 50 | 45 | 50 |
| Earliness | 40 | 50 | 40 |
| Setting | 70 | 70 | 70 |
| Flower sex | M | M | M |
| Rind color | Tiger | Crimson dark | Crimson dark |
| No. fruit/plant⁶ | 3 | 3 | 3 |
| Fruit shape | 50-60 (round to | 50 (round) | 50 (round) |
| | oval) | | |
| Fruit striping | 25² | 80¹ | 70³ |
| Fruit regularity | 80 | 80 | 80 |
| Fruit weight (Kg) | 2 | 3 | 3 |
| Flesh color | Deep red | Deep red | Deep red |
| Flesh firmness | 70 | 75 | 75 |
| Rind thickness | 5 | 12 | 10 |
| (mm) | | | |
| Rind Crack | 10 | 10 | 10 |
| Seed color | Brown dark | Brown | Seedless |
| TSW⁵ (gr) | 24 | 60 | Seedless |
| Seed size (mm) | 1.1*0.4 (oval) | 6*9 | Seedless |
| Taste | Good | Good | Good |
| TSS (Brix %) | 13 | 11 | 11-13 |
| PMW1 | 9 | 2 | 7 |
| PMW2 | 8 | 2 | 6 |

*PMI: Tolerance 6-7 (also referred to as intermediate resistance)
¹80% indicating Gray rind with wide dark green stripes that cover about 80% of the fruit's rind;
²25% indicating Gray rind with thin dark green stripes that cover about 25% of the fruit's rind;
³70% indicating Gray rind with wide dark green stripes that cover about 70% of the fruit's rind;

### Example 3: Further Productions of Triploid Seedless Watermelon with PM Resistance—Development Stages

### 12.702b Pathway:

Summer 2008: A cross have been taken with two Origene Seeds proprietary lines: “237a4” being PM sensitive (PMS) and “406” being PM resistant (PMR, described hereinabove) to create the line “554-F1”.
Summer 2010: A backcross between “554-F5” and Origene Seeds proprietary line “112” (described hereinbefore) was made to create “572-F1”.
Winter 2012: A cross between the diploid line “572-b-F5” (PMR) as a male and Origene Seeds Proprietary tetraploid line “612a” (PMS) as a female to create the triploid hybrid “12.702b”. This hybrid was created also in winter 2014 with “572-b-F9” and “612a” and was deposited under NCIMB42359.

### 12.135b Pathway:

Summer 2007: A cross have been made with two Origene Seeds proprietary lines: “ZGM” being PM sensitive (PMS) and “406” being PM resistant (PMR) to create the line “538-F1”.
Winter 2010: A second backcross have been made between “538v2-F7” and Origene Seeds proprietary line “523t-F7” to create the male line “675-F1”.
Winter 2014: A cross between the diploid male line “675-b2-F8” (PMR) and the Origene Seeds proprietary female line “642b11-1-F12” (PMS) to create the hybrid “12.135b”.

### Example 4: Sequence Related Amplification Polymorphism (SRAP)

### Analysis:

In order to investigate heritage of the tolerance or resistance to PM from the male parent to the triploid hybrid watermelon plant, the conservation of the trait locus among the various watermelon male parent was analyzed using the SRAP technology and various combinations of primer pairs known in the art (see Table 2 in Levi et al. 2006).

The amplified DNA fragments were separated by denaturing acrylamide gels and detected by autoradiography. Among the various combinations, the combination me1em1 identified a 480 bp locus in all the male parents tested, including W11-701-1, 572 (W11-768-B) and 576-a-13-2-8B1 (as well as in the other, male diploids disclosed herein) but was missing from cultivated male diploid watermelon plants sensitive to PM.

What is claimed is:

1. A triploid hybrid watermelon plant characterized by a resistance to powdery mildew (PM) race 1 and race 2 as quantified by a tolerance of less than about 3.5 on the Tetteh scale, and having properties such that seedless fruits are produced with a total soluble solid (TSS) of at least 10%, flesh firmness of between 60 and 80 on a scale of 10 to 90, where 10 indicates very soft flesh and 90 indicates a very firm flesh, and a flesh color selected from the group consisting of yellow, orange or red fruit flesh color, preferably dark red.

2. The triploid hybrid watermelon of claim 1, carrying genetic background, linked to resistance to PMW1 and PMW2, of a wild type watermelon PI 482312.

3. (canceled)

4. (canceled)

5. The triploid hybrid watermelon plant of claim 1, characterized by a number of fruits per plant or 2-5.

6. The triploid hybrid watermelon plant of claim 1, characterized by a vigor of between 50 to 80.

7. The triploid hybrid watermelon plant of claim 1, characterized by a fruit setting of between 20 to 50.

8. The triploid hybrid watermelon plant of claim 1, characterized by rind crack of between 10 to 30.

9. The triploid hybrid watermelon of claim 1, having an essentially round/oval fruit and a fruit weight between 2-10 kg.

10. (canceled)

11. (canceled)

12. The triploid hybrid watermelon plant of claim 1, wherein said resistance to PMW1 and PMW2 is genetically linked to at least one marker locus which co-segregates with PM resistance trait and can be identified in a PCR reaction by amplification of a DNA fragment with a pair of PCR oligonucleotide primers represented by a forward primer of SEQ ID NO:1 and a reverse primer of SEQ ID NO:2.

13. The triploids hybrid watermelon plant of claim 1, designated herein: S12-12.701 having representative seeds deposited under Accession No. NCIMB42044; 12.704bPMI-W12 having representative seeds deposited under Accession No. NCIMB42172; 12.702b-W14 having representative seeds deposited under Accession No. NCIMB42359 and 12.135b-W14 having representative seeds deposited under Accession No. NCIMB42360.

14. A progeny of a triploid watermelon plant of claim 1, the progeny maintaining at least an intermediate resistance to PMW1 and PMW2.

15. The progeny of claim 14, being asexually reproduced from the triploid watermelon plant of claim 1.

16. The progeny of claim 14, of a triploid watermelon selected from any one of the plants having representative seeds deposited under Accession Nos. NCIMB42044, NCIMB42083, NCIMB42172, NCIMB42359 and NCIMB42360.

17. A seedless watermelon fruit of a plant of claim 1 or of a progeny of claim 14.

18. A triploid hybrid watermelon plant characterized by a resistance to powdery mildew (PM) race 1 (PMW1) and race 2 (PMW2) as quantified by a resistance of less than about 3.5 on the Tetteh scale, and having properties such that seedless fruits are produced with a total soluble solid (TSS) of at least 10%, said resistance to PMW1 and PMW2 is genetically linked to at least one marker locus which co-segregates with PM resistance trait and can be identified in a PCR reaction by amplification of a DNA fragment with a pair of PCR oligonucleotide primers represented by a forward primer of SEQ ID NO:1 and a reverse primer of SEQ ID NO:2.

19. The watermelon plant of claim 18, further comprising a watermelon plant and seed, namely of Citrullus lanatus, which are tolerant to powdery mildew (PM) race 1 (PMW1) and race 2 (PMW2), comprising in their genome introgressed sequences from Citrullus lanatus var. Citroides conferring said PM resistance.

20. The watermelon plant of claim 18, further comprising a resistance locus genetically linked to at least one marker locus which co-segregates with PM resistance trait and comprising a marker that identified for the resistance to powdery mildew (PM) race 1 (PMW1) and race 2 (PMW2).

21. The watermelon plant of claim 20, wherein said resistance trait is conferred by an introgression of genomic DNA of Citrullus lanatus var. Citroides on the chromosome 2 of Citrullus lanatus.

22. The watermelon plant of claim 21, wherein said introgression of Citrullus lanatus var. Citroides into the Citrullus lanatus genome is substantially between nucleotides 25,000,000-26,200,000.

23. The watermelon plant of claim 21, wherein said introgression of Citrullus lanatus var. Citroides into the Citrullus lanatus genome is substantially between nucleotides 25,000,000-25,900,000.

24. The watermelon plant of claim 21, wherein said introgression of Citrullus lanatus var. Citroides into the Citrullus lanatus genome is substantially between nucleotides 25,900,000-26,100,000.

25. The watermelon plant of claim 21, wherein said introgression of Citrullus lanatus var. Citroides into the Citrullus lanatus genome is substantially between nucleotides 26,100,000-26,200,000.

26. A diploid, triploid or tetraploid watermelon plant characterized by having tolerance to PM and seed yield of at least 4 within a score scale from 1 to 9.

27. A Citrullus lanatus seed for producing a hybrid watermelon plant characterized by a resistance to powdery mildew (PM) race 1 and race 2 as quantified by a tolerance of less than about 3.5 on the Tetteh scale, and having properties such that seedless fruits are produced with a total soluble solid (TSS) of at least 10%, and a flesh color selected from the group consisting of yellow, orange or red fruit flesh color, preferably dark red, said seed having in its genome introgressed sequences from Citrullus lanatus var. Citroides conferring tolerance to fungi wherein said introgressed sequences are located on chromosome 2, within the chromosomal region delimited by nucleotides 25,000,000-26,200,000.

28. A Seed according to claim 27, wherein said resistance is a high tolerance or at least intermediate resistance to powdery mildew (PM) race 1 (PMW1) and race 2 (PMW2).

29. A Seed according to claim 28, wherein said seed is diploid, triploid, tetraploid and/or allopolyploid.

30. A Citrullus lanatus plant resistant to fungi grown from a seed according to claim 29.

31. A Hybrid plant of Citrullus lanatus, obtainable by crossing a plant resistant to fungi according to claim 30 with a susceptible plant of C. lanatus.

32. A method for detecting and/or selecting C. lanatus plants having introgressed sequences from Citrullus lanatus var. Citroides wherein said introgressed sequences conferring tolerance to fungi, comprising detection of a marker selected from the group consisting of a marker in the 3′ region of the introgression, marker in the middle of the introgression and a marker in the 5′ region of the introgression, in a genetic material sample of the plant to be selected.

33. The watermelon plant of claim 19, wherein said C. lanatus plants further includes a selection of a triploid watermelon hybrid devoid of only one parent is tolerant to PM.

34. The method for detecting and/or selecting C. lanatus plants having introgressed sequences from Citrullus lanatus var. Citroides of claim 29, wherein said method further includes a selection of a triploid watermelon hybrid devoid of only one parent is tolerant to PM.

35. The watermelon plant of claim 18 further comprising:
a) a resistance locus genetically linked to at least one marker locus which co-segregates with PM resistance trait and comprising a marker identified for the resistance to PM fungus;
b) wherein said resistance trait is conferred by an introgression of genomic DNA of Citrullus lanatus var. Citroides on the chromosome 2 of Citrullus lanatus;
c) wherein a reproduction of said Citrullus lanatusis characterized by a cutting off of a part of the Citrullus lanatus embryos.

36. A seed according to claim 27, characterized by a reproduction of diploid, tetraploid or triploid offspring by a technique selected from the group consisting of a cutting, a tissue culture, an embryo rescue, a chromosome doubling and double haploids and mutations.

37. The watermelon plant of claim 18 comprising at least one watermelon fruit of Citrullus lanatus, resistant to fungi, especially to fungi including PM, including a genome introgressed sequences from Citrullus lanatus var. Citroides conferring resistance to said fungi.

38. The watermelon plant of claim 22, wherein said watermelon plant is a triploid plant.

39. The watermelon plant of claim 35, wherein said fruit is seedless.

40. The watermelon plant of claim 35, wherein said fruit has a substantially yellow, orange or red fruit flesh color, preferably dark red flesh color.

41. The watermelon plant according to claim 22, wherein said introgressed sequences are sequenced by way of High Throughput Sequencing (HTS) or Next Generation Sequencing (NGS), thereby readily facilitating sequencing the entire genome substantially at once.

42. The watermelon plant according to claim 22, wherein said HTS includes a next-generation “short-read” and a third-generation “long-read” sequencing method, which said sequencing is selected from the group consisting of an exome sequencing, a genome sequencing, a genome resequencing, a transcriptome profiling (RNA-Seq), a DNA-protein interactions (ChIP-sequencing), and an epigenome characterization

43. A watermelon plant characterized by:
resistance to Watermelon powdery mildew (PM);
seedless fruit having a total soluble solid (TSS) of at least 10%;
being a diploid, triploid or tetraploid plant; and
yellow, orange or red fruit flesh color, preferably dark red fruit flesh color.

44. The watermelon plant of claim 26, designated herein 12.702b-W14, having representative seeds deposited under Accession No. NCIMB42359.

45. The watermelon plant of claim 26, designated herein 12.135b-W14, having representative seeds deposited under Accession No. NCIMB42360.

46. A diploid watermelon capable of pollinating a tetraploid watermelon to produce a triploid hybrid watermelon tolerant to powdery mildew (PM) and capable of producing seedless fruits with a total soluble solid (TSS) of at least 10%.

47. (canceled)

48. The diploid, triploid or tetraploid watermelon plant of claim 26, having an essentially round/oval fruit and a fruit weight between 2-10 kg.

49. (canceled)

50. (canceled)

51. The diploid, triploid or tetraploid watermelon plant of claim 1, having an essentially round/oval fruit and a fruit weight of between 2 to 10 kg.

52. The watermelon plant of claim 21, wherein said introgression of Citrullus lanatus var. Citroides into the Citrullus lanatus genome is substantially between nucleotides 24,000,000-28,000,000.

53. A Citrullus lanatus seed for producing a hybrid watermelon plant characterized by a resistance to powdery mildew (PM) race 1 and race 2 as quantified by a tolerance of less than about 3.5 on the Tetteh scale, and having properties such that seedless fruits are produced with a total soluble solid (TSS) of at least 10%, and a flesh color selected from the group consisting of yellow, orange or red fruit flesh color, preferably dark red, said seed having in its genome introgressed sequences from Citrullus lanatus var. Citroides conferring tolerance to fungi wherein said introgressed sequences are located on chromosome 2, within the chromosomal region delimited by nucleotides 24,000,000-28,000,000.
